(19) United States
(12) Patent Application Publication
(10) Pub. No.: US 2026/0315008 A1
(43) Pub. Date: Oct. 15, 2026

(54) SYSTEMS AND METHODS FOR CARDIOVASCULAR DISEASE DETECTION

(71) Applicant: The Chinese University of Hong Kong, Hong Kong (CN)

(72) Inventors: Ni ZHAO, Hong Kong (CN); Shirong QIU, Meizhou (CN)

(21) Appl. No.: 19/175,971

(22) Filed: Apr. 10, 2025

(51) Int. Cl. A61B 5/00; A61B 5/02; A61B 5/0205; A61B 5/021; A61B 5/022; A61B 5/024; A61B 5/0245; A61B 5/053; A61B 5/11; A61B 5/346; G16H 50/70

(52) U.S. Cl. CPC A61B 5/7275; A61B 5/02007; A61B 5/02028; A61B 5/0205; A61B 5/02125; A61B 5/02208; A61B 5/02225; A61B 5/02416; A61B 5/0245; A61B 5/053; A61B 5/1102; A61B 5/346; A61B 5/4088; A61B 5/7264; A61B 5/742; A61B 5/746; G16H 50/70; A61B 2562/0247

(57) ABSTRACT

Disclosed is a system and a method for assessing risk and/or severity of a cardiovascular disease in a subject, by determining, from data from a blood pressure measurement and/or a cardiac rhythm measurement in the subject, a plurality of stroke volume allocation (SVA) values and assessing risk and/or severity of the cardiovascular disease based on the SVA values and/or one or more cardiovascular parameters derivable from the SVA values. An SVA value represents a ratio of a stroke volume stored in the aorta of the subject during the systole phase in a cardiac cycle to a total stroke volume pumped from the heart of the subject during the cardiac cycle.

### TECHNICAL FIELD

The present application generally relates to the field of medicine, and in particular, to systems and methods for cardiovascular disease detection, e.g., for assessing risk and/or severity of a cardiovascular disease in a subject by analyzing blood pressure measurements and/or cardiac rhythm measurements in the subject.

### BACKGROUND OF THE INVENTION

Population aging has become an increasingly critical issue in both developed and developing countries in recent years, and it calls for fundamental changes in existing models of healthcare and social support. About 80% of all cardiovascular mortality occurs in low-income and middle-income countries, where people often do not benefit from primary health care programs. In particular, to reduce the economic and societal burden of medical treatment for cardiovascular diseases associated with aging, it is highly desired to deploy mobile health monitoring devices to individuals, thus allowing prognosis of diseases and early medical intervention. Current commercially available or lab-demonstrated wearable devices are capable of providing a variety of techniques to routinely monitor our basic vital signs, such as electrocardiogram (ECG) for heart rate monitoring and atrial fibrillation detection, phonocardiography (PCG) for heart rate monitoring and also diagnosis of heart valve diseases, and photoplethysmography (PPG) for heart rate and blood oxygen level (SpO₂) monitoring. However, current wearable devices are unable to reveal certain critical cardiovascular-related medical information hidden in the sensor signals, which is one of the fundamental functional limitations of current medical wearables and constrains off-the-shelf sensor applications, and therefore is detrimental to the efficient use of global medical resources. As such, there exists an urgent need for developing new and effective methods and systems useful in cardiovascular disease assessment. The present application fulfills this and other related needs.

### BRIEF SUMMARY OF THE INVENTION

In a first aspect, provided is a system (e.g., a medical system or a wearable device (e.g., a smart watch)) for assessing risk and/or severity of a cardiovascular disease in a subject, comprising:
a blood pressure measuring device and/or a cardiac rhythm monitor, wherein the blood pressure measuring device is configured to perform a blood pressure measurement on the subject, and the cardiac rhythm monitor is configured to perform a cardiac rhythm measurement on the subject;
a processor configured to (i) determine, from data from the blood pressure measurement and/or the cardiac rhythm measurement in the subject, a plurality of stroke volume allocation (SVA) values, (ii) calculate an average SVA value from the plurality of SVA values and compare the average SVA value with a reference SVA value derived from a population of healthy individuals, and (iii) for an average SVA value which is below the reference SVA value, generate an assessment result indicating that the subject has an increased risk and/or severity of the cardiovascular disease as compared with the healthy individuals; and
an output device (e.g. a display) for outputting the assessment result;
wherein an SVA value represents a ratio of a stroke volume stored in the aorta of the subject during the systole phase in a cardiac cycle to a total stroke volume pumped from the heart of the subject during the cardiac cycle.

In some embodiments of the first aspect, the data is from a blood pressure measurement and comprises a blood pressure waveform in the cardiac cycle. In some particular embodiments, an SVA value is generated by:
i) calculating the area under the blood pressure waveform during the diastolic phase of the cardiac cycle, defined as A_{d};
ii) calculating the area under the blood pressure waveform during the systolic phase of the cardiac cycle, defined as Aₛ; and
iii) calculating the SVA value by the following equation:

$$S V A = \frac{Ad}{As + Ad} .$$

In some embodiments, the blood pressure waveform comprises a first valley point, a dicrotic notch point and a second valley point. The systolic phase is a phase substantially between the first valley point and the dicrotic notch point. The diastolic phase is a phase substantially between the dicrotic notch point and the second valley point. In some embodiments, Aₛ is obtained by calculating the area under the blood pressure waveform from the first valley point to the dicrotic notch point. In some embodiments, A_{d} is obtained by calculating the area under the blood pressure waveform from the dicrotic notch point to the second valley point.

In some embodiments of the first aspect, the data is from a blood pressure measurement and a cardiac rhythm measurement, and comprises a blood pressure waveform and a pulse waveform in the cardiac cycle. In some particular embodiments, and an SVA value is generated by:
a) extracting a diastolic blood pressure (DBP) and a mean blood pressure (MBP) from the data, and extracting a cardiac duration and a systolic duration from the pulse waveform; and
b) calculating the SVA value by the following equation:

$$S V A {=}^{Δ} ( δ + Δ {δ}_{pulse} ) \cdot ( 1 - \frac{Ts}{Tc} ) ,$$

wherein Tₛ is the systolic duration, T_{c} is the cardiac duration,

$$δ = \frac{D B P + M B P}{2 \cdot D B P} , and {Δδ}_{pulse} = \frac{{Ad}_{testing} - {Ad}_{resting}}{{Ad}_{resting}} .$$

The Ad_{testing} is the area under a pulse waveform during the diastolic phase of the cardiac cycle when the subject is at a test status, and the Ad_{resting} is the area under a pulse waveform during the diastolic phase of a cardiac cycle when the subject is at a resting status.

In some particular embodiments, the test status is selected from the group consisting of a resting status, a status of vasodilation, a status of vasoconstriction and any combination thereof.

In some embodiments of the first aspect, the processor is further configured to:
(1) by using the steps a) and b) above, generate a plurality of pairs of blood pressure (BP) and SVA values and calculating an average δ at a plurality of first timepoints, wherein a single blood pressure waveform and a single pulse waveform obtained at the same timepoint are used to determine each pair of BP and SVA values,
(2) determine bs*, ks*, PSR and b by feeding the plurality of pairs of BP and SVA values into a formula for least square method to minimize an error in BP as follows:

$${arg min}_{{b}_{s}^{*} , b , PSR , {k}_{s}^{*}} ‖ BP - \frac{{b}_{s}^{*} - S V A \cdot b}{S V A \cdot ( PSR - 1 ) + 1 - {k}_{s}^{*}} ‖ ,$$

(3) generate SVA values at a plurality of second timepoints from a pulse waveform in the cardiac cycle by the following equation:

$$S V A {=}^{Δ} ( δ + Δ {δ}_{pulse} ) \cdot ( 1 - \frac{Ts}{Tc} ) ,$$

wherein Tₛ, T_{c}, and Δδₚᵤₗₛₑ are defined as in above paragraphs and δ is the average δ calculated in step (1); and
(4) calculate BP values through the SVA values at the plurality of second timepoints according to the following equation:

$$BP - \frac{{b}_{s}^{*} - S V A \cdot b}{S V A \cdot ( PSR - 1 ) + 1 - {k}_{s}^{*}} .$$

In some embodiments of the first aspect, the blood pressure measurement and/or the cardiac rhythm measurement is performed on a peripheral artery and a central artery, respectively. In some particular embodiments, the processor is further configured to:
i) determine, from data from the blood pressure measurement and/or the cardiac rhythm measurement in the subject, a first SVA value for the peripheral artery and a second SVA value for the central artery;
ii) determine RatioSVA as a ratio of the first SVA value to the second SVA value, and
iii) assess arterial stiffness with the RatioSVA.

An increase in the RatioSVA value in the subject as compared with a reference RatioSVA value derived from a population of healthy individuals may indicate a higher degree of arterial stiffness in the peripheral artery of the subject than the population of healthy individuals.

In some embodiments of the first aspect, the processor is further configured to determine a probability of the cardiovascular disease by the following equation:

p(x)=1−1/(1+e^{-(β}^{₀}^{+β}^{₁}^{x}^{₁}^{+β}^{₂}^{x}^{₂}^{+β}^{₃}^{x}^{₃}^{+β}^{₄}^{x}^{_{4+β}}^{₅}^{x}^{₅}⁾),

wherein X₁ is a heart rate (HR), X₂ is a left ventricular ejection time (LVET), X₃ is a systolic blood pressure (SBP), X₄ is a diastolic blood pressure (DBP), X₅ is a SVA value, β₀=−92.7477, β₁=0.3109, β₂=41.5207, β₃=0.0627, β₄=−0.1047, β₅=0.9826. An increase in the probability in the subject as compared with a reference p(x) value derived from a population of healthy individuals may indicate that the subject has a risk of developing the cardiovascular disease.

In some embodiments of the first aspect, the data is from a blood pressure measurement and a cardiac rhythm measurement, and the processor is further configured to assess an organ damage induced by a pulsatile energy in the cardiovascular system of the subject with a systemic reflection coefficient (SRC) value calculated by the following equation:

$$S R C = 2 \frac{S V A}{\frac{( {e}^{\frac{{T}_{c} - {T}_{s}}{τ}} - 1 ) \cdot D B P}{P P}} - 1 ,$$

wherein DBP is a diastolic blood pressure, PP is a pulse pressure, Tₛ is a systolic duration from a pulse waveform, T_{c} is a cardiac duration from the pulse waveform, and wherein DBP and PP are from the blood pressure measurement, Tₛ and T_{c} are from the cardiac rhythm measurement, and t is calculated with DBP, PP, Ts and Tc by the following equation:

$$\frac{D B P}{P P} = \frac{{e}^{\frac{- {T}_{s}}{τ}} \cdot \frac{1}{{( \frac{1}{τ} )}^{2} + {( \frac{π}{{T}_{s}} )}^{2}} [ ( {e}^{\frac{{T}_{s}}{τ}} + 1 ) {( \frac{π}{{T}_{s}} )}^{2} ]}{2 \cdot ( {e}^{\frac{{T}_{c} - {T}_{s}}{τ}} - {e}^{\frac{- {T}_{s}}{τ}} )} .$$

An increase in the SRC value in the subject as compared with a reference SRC value derived from a population of healthy individuals can indicate an organ damage in the subject.

In some particular embodiments, the processor is further configured to assess the cardiovascular disease with indicators derived from the SRC, the SVA, a mean blood pressure (MBP), and the PP as follows:

NormI= [symbol] · [symbol] / [symbol] .

PASI= [symbol] / [symbol] ;

PODI= [symbol] / [symbol] · [symbol] ;

HRMDI= [symbol] · [symbol] · [symbol] ;

HZDI=1/( [symbol] · [symbol] );

wherein the symbol ~ for each parameter represents a ratio of a value measured for the subject to a reference value derived from a population of healthy individuals.

In some embodiments, an increase in NormI in the subject from a first timepoint to a second timepoint indicates an improvement in the cardiovascular system of the subject from a first timepoint to a second timepoint.

In some embodiments, an increase in PASI in the subject from a first timepoint to a second timepoint indicates a higher risk for arterial stiffness due to poor cushioning function and a higher SRC in the subject at the first timepoint than that at the second timepoint.

In some embodiments, an increase in PODI in the subject from a first timepoint to a second timepoint indicates a higher risk for pulsatile-induced organ damage due to poor pulsation reflexes, poor cushioning function, and a large pulsation component of BP in the subject at the first timepoint than that at the second timepoint.

In some embodiments, an increase in HRMDI in the subject from a first timepoint to a second timepoint indicates a higher risk for systemic-vascular-resistance-induced microcirculation damage in the subject at the first timepoint than that at the second timepoint.

In some embodiments, an increase in HZDI in the subject from a first timepoint to a second timepoint indicates a higher risk for hazardous cardiovascular system with arterial stiffness and pulsatile-induced organ damage in the subject at the first timepoint than that at the second timepoint.

The cardiovascular disease may be selected from the group consisting of mild cognitive impairment, stroke, dementia, valvular aortic stenosis, aortic incompetence and any combination thereof.

In some embodiments of the first aspect, the assessment result comprises an alert about the risk and/or severity of the cardiovascular disease, and/or a treatment recommendation.

In some embodiments of the first aspect, the system further comprises a memory configured to store data from the blood pressure measurement and/or the cardiac rhythm measurement, the reference SVA value and/or the assessment result.

In some embodiments of the first aspect, the blood pressure measuring device is selected from the group consisting of a pressure sensor, a digital sphygmomanometer, stethoscope sphygmomanometer and any combination thereof.

In some embodiments of the first aspect, the cardiac rhythm monitor is selected from the group consisting of a cardiac rhythm monitor based on electrocardiogram (ECG), a cardiac rhythm monitor based on photoplethysmography (PPG), a cardiac rhythm monitor based on phonocardiography (PCG), a cardiac rhythm monitor based on impedance cardiography (ICG), a cardiac rhythm monitor based on ultrasound, a cardiac rhythm monitor based on ballistocardiography (BCG), a cardiac rhythm monitor based on seismocardiographic (SCG) and any combination thereof.

In some embodiments of the first aspect, the blood pressure measuring device and the cardiac rhythm monitor are configured to make simultaneous measurements.

In a second aspect, provided is a method for assessing risk and/or severity of a cardiovascular disease in a subject, comprising:
generating data from a blood pressure measurement and/or a cardiac rhythm measurement in the subject;
determining from the data a plurality of stroke volume allocation (SVA) values;
calculating an average SVA value from the plurality of SVA values and comparing the average SVA value with a reference SVA value derived from a population of healthy individuals;
for an average SVA value which is below the reference SVA value, generating an assessment result indicating that the subject has an increased risk and/or severity of the cardiovascular disease as compared with the healthy individuals; and
outputting the assessment result;
wherein an SVA value represents a ratio of a stroke volume stored in the aorta of the subject during the systole phase in a cardiac cycle to a total stroke volume pumped from the heart of the subject during the cardiac cycle.

In a third aspect, provided is a computer-readable storage medium having stored therein program code instructions that, when executed by a processor in a computer system, cause the processor to perform a method in the second aspect.

In a fourth aspect, provided is a computer product comprising a non-transitory computer readable medium storing a plurality of instructions that when executed cause a computer system to perform a method in the second aspect.

In a fifth aspect, provided is a system comprising the computer product described in the fourth aspect and one or more processors for executing instructions stored on the computer readable medium.

In a sixth aspect, provided is a system comprising means for performing a method in the second aspect.

In a seventh aspect, provided is a system comprising one or more processors configured to perform a method in the second aspect. These and other embodiments of the disclosure are described in detail below. For example, other embodiments are directed to systems, computer products, and computer readable media associated with the method in the second aspect.

A better understanding of the nature and advantages of embodiments of the present disclosure may be gained with reference to the following detailed description and the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram illustrating an exemplary system for cardiovascular disease detection in the present application.

FIG. 2 illustrates an exemplary configuration that enables the functionality of the system for cardiovascular disease detection in the present application.

FIG. 3 is a schematic diagram showing concepts of arterial cushioning function and the pulsatile energy-induced organ damage in the human cardiovascular system. (a) Cushioning function of the cardiovascular system. The elastic artery (e.g., aorta) performs a Windkessel effect to buffer volume/energy that pumps from the heart during the systolic phase. In the diastolic phase, the valve is closed, and the remainder volume stored in the elastic artery would be pushed into peripheral arteries and organs. (b) pulsatile energy-induced organ damages in the human cardiovascular system. (c) the vital organs that may associate with potential damages caused by the excess pulsatile energy in the circulation.

FIG. 4 shows exemplary principles and models of the stroke-volume-allocation (SVA) and systemic reflection coefficient (SRC). In FIG. 4, C denotes the arterial compliance, P denotes the blood pressure, R denotes the systemic vascular resistance, Zc denotes the characteristic impedance, I(t) denotes blood flow pumping out of the heart, and Ic(t) denotes blood flow buffered by arterial compliance C. In addition, in the BP waveform boxes, PP is the pulse pressure, DBP is the diastolic blood pressure and τ is the time constant in the diastolic phase of the BP waveform. (a) SVA is influenced by the systolic and diastolic processes of the circulation and can be calculated from the area ratio of the blood pressure waveform, i.e., SVA=A_{d}/(Aₛ+A_{d}). The classical two-element Windkessel model is constructed from total arterial compliance (C) and systemic vascular resistance (SVR) with blood pressure (P) as the power source; arterial compliance (C) buffers blood volume primarily (during systole of duration Ts) and then provides sufficient energy to the organs during diastole phase (duration Tc-Ts). (b) The SRC is related to impedance matching of the characteristic impedance and the systemic vascular resistance and can be calculated from SVA with additional time decay constant. The three-element Windkessel model is constructed from the total arterial compliance (C) and systemic vascular resistance (SVR), and characteristic impedance (Zc) under blood pressure (P) as the power source. Different from the two-element Windkessel model for buffer function evaluation, the additional characteristic impedance (Zc) helps to evaluate vascular impedance mismatching between the Zc and SVR and as a function of the time constant of an ideal BP wave.

FIG. 5 shows an exemplary system flowchart for SVA and SRC measurements in arterial system risk assessment, including arterial stiffness assessment, blood pressure tracking, cardiovascular abnormality detection, and vital organ damage assessment according to some embodiments of the present application.

FIG. 6 shows diverse pulse wave-based cushioning functions of arteries according to some embodiments of the present application. (a) Different scenarios of arterial conditions, such as normal (e.g., when the subject is at resting status), vasodilation, and vasoconstriction. (b) diverse SVA values for different arterial conditions. (c) examples of peripheral arterial stiffness in arm, hand, leg and foot. (d) ratio of the SVA between the central (SVAc) and peripheral (SVAp) arteries. (e) SVA values at a normal status (e.g., when the subject is at resting status) v.s. at a stiffening status (e.g., when the subject is exercising).

FIG. 7 shows exemplary SVA calculation based on combining the input cardiac signals from off-the-shelf pulse sensors and blood pressure according to some embodiments of the present application.

FIG. 8 shows an exemplary SVA-BP model for cuffless beat-by-beat BP tracking according to some embodiments of the present application.

FIG. 9 shows an exemplary SVA-cardiovascular disease model (SVA-CVD model) for CVD abnormality diagnosis according to some embodiments of the present application.

FIG. 10 shows an exemplary SRC calculation for potential organ damage assessment according to some embodiments of the present application.

FIG. 11 shows an exemplary SRC-organ damage model (SVA-OD model) for extraction of cardiovascular system (CVS) indicators according to some embodiments of the present application.

FIG. 12 shows SVA validation in open clinical datasets according to some embodiments of the present application. (a) Dynamic SVA v.s. dynamic Ts/Tc, where Ts is the systolic duration, Tc is the heartbeat cycle. (b) Significant inverse relationship between SVA and carotid-femoral pulse wave velocity (cfPWV), which implies that SVA can be applied to AS assessment. (c) Significant SVA differences among the normal individuals, individuals with valvular aortic stenosis (VAS), and individuals with aortic incompetence (AI), which indicated SVA can be applied to cardiovascular disease-related heart damage (CHD) assessment. (d) An example of long-term blood pressure (BP) tracking (>20 hours) based on the SVA-BP model. (e) The ratio of DBP to MBP in individuals.

FIG. 13 shows an illustration of an exemplary application of off-the-shelf sensors in SVA measurement according to some embodiments of the present application. (a) Pressure, ECG, PCG, and PPG sensors for cardiac monitoring in the same subject. (b) SVA trend extracted by the different sensors. (c) SVA accuracy evaluation in the different sensors.

FIG. 14 shows exemplary SVA measurement using off-the-shelf wearable devices according to some embodiments of the present application. (a) SVA evaluation through ECG-based monitoring performed through Apple Watch measurements and PPG-based monitoring performed through an INTS finger ring. (b) Correlations of vascular aging with SVA and cfPWV. Error bars denote s.d. from the mean. (c) cfPWV and SVA for patients with percutaneous coronary intervention (PCI) (34 patients) and normal individuals (14 normal individuals). (d, e) SVA-BP tracking and accuracy for the PPG-based INTS finger ring. (f) Areas under the curve, i.e., AUCs, of various methods for distinguishing normal from poor arterial systems (i.e., 41 individuals without CHD, and 104 individuals with CHD). (g) Validation of p(x) scores derived from SVA measurements in determining probability of cardiovascular diseases.

FIG. 15 shows exemplary SRC-OD measurement for assessments of different diseases according to some embodiments of the present application. (a) SRC-OD measurement interprets different patterns of normal subjects, patients with valvular aortic stenosis (VAS), patients with aortic incompetence (AI), and patients with chronic renal failure (CRF). (b) SRC-OD measurement interprets different patterns of normal subjects and patients with mild cognitive impairment (MCI) at the same age group.

FIG. 16 shows exemplary SRC measurements for assessments of organ damages according to some embodiments of the present application.

### DETAILED DESCRIPTION OF THE INVENTION

All publications, patents, and patent applications mentioned in this specification are incorporated herein by reference to the same extent as if each individual publication, patent, or patent application had been specifically and individually indicated to be incorporated by reference.

Before the present disclosure is described in detail below, it is to be understood that the present disclosure is not limited to the specific methodologies, protocols, and reagents described herein, as these may vary. It is also to be understood that the terms used herein are for the purpose of describing particular embodiments only and are not intended to limit the scope of the present disclosure. Unless defined otherwise, all technical and scientific terms used herein have the same meaning as commonly understood by one of ordinary skill in the art to which the present disclosure pertains.

Certain embodiments disclosed herein include numerical ranges, and certain aspects of the present disclosure may be described in terms of ranges. Unless otherwise indicated, it is to be understood that the numerical ranges or the manners described in terms of ranges are for purposes of brevity and convenience only, and are not to be considered as a strict limitation on the scope of the present disclosure. Accordingly, the description in terms of ranges should be considered to specifically disclose all possible sub-ranges and all possible specific numerical points within that range, as if such sub-ranges and numerical points had been expressly written herein. The above principles apply equally regardless of the width of the numerical values. When description is made by using a range, the range includes the endpoints of the range.

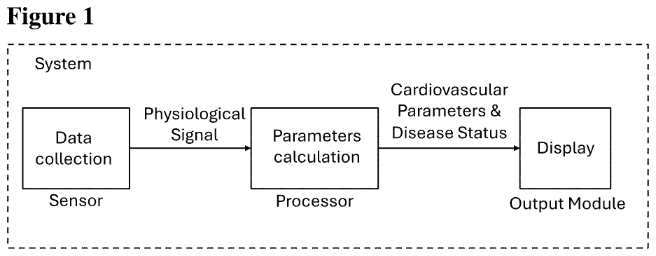

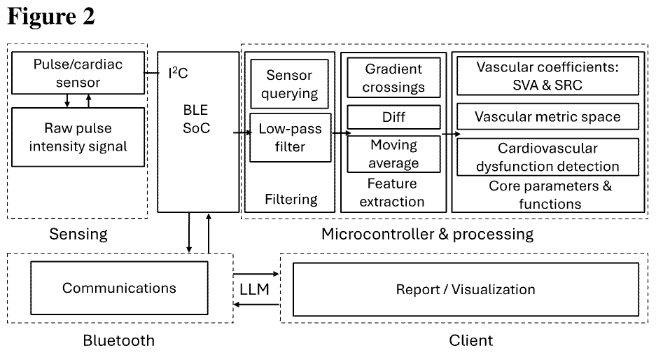

The global wearable medical device market size was worth USD 21.3 billion in 2021 and estimated to be growing at a 28.1% compound annual growth rate (CAGR) from 2022 to 2030. The market is showcasing evident potential in the current context. The most popular wearable devices are smart watches and fitness bands, of which a selling and feature point is their ability to track the users' health condition noninvasively and unobtrusively. Most of these devices on market like Apple watch, LG Watch Urbane, Motorola Moto 360 smart watch, Huawei Watch, Microsoft Band, use optical sensors and/or electrical sensors to acquire PPG signals and/or ECG signals for monitoring multi-physiological parameters like heart rate, respiration rate, blood pressure and so on. However, these commercial wearable devices still could not have a comprehensive function in monitoring the risk of arterial wall and remains unsatisfactory for wide user acceptance. The methods and systems of the present application can be implemented in these wearable platforms and applying existing hardware in a new way (see e.g., FIGS. 1 and 2).

FIG. 1 illustrates an exemplary system comprising at least one sensor (e.g., a blood pressure measuring device and/or a cardiac rhythm monitor), a processor, and an output module. The sensor is configured to acquire a physiological data, such as a pulse wave or a cardiac signal, and transmit it to the processor. The processor is communicatively coupled to the sensor and is configured to compute a series of cardiovascular-related physiological parameters from the physiological signal and to assess the disease state based on these parameters. The output module is configured to receive an assessment result generated from the processor, and/or display or transmit the assessment result.

FIG. 2 illustrates an exemplary configuration that enables the functionality of the system for cardiovascular disease detection in the present application. The system shown in FIG. 2 comprises a sensing module, a processor (e.g., a microcontroller), an output module (e.g., a Bluetooth). The sensing module can acquire physiological signals (such as a pulse wave, a cardiac signal, and/or a raw pulse intensity signal) from a subject. The acquired signals can be transmitted to the processor for processing. The processor can perform (i) a preprocessing on the acquired signals to reduce noises in the acquired signals, e.g., by sensor querying and/or low-pass filter; (ii) a feature extraction on the acquired signals and/or the preprocessed signals, to extract features (such as diastolic blood pressure (DBP), mean blood pressure (MBP), systolic duration (Tₛ), cardiac duration (T_{c}), heart rate (HR), left ventricular ejection time (LVET), etc.) from the signals; (iii) calculate cardiovascular-related physiological parameters (such as the SVA values, the RatiosVA value, the BP value calculated with the SVA values, the p(x) value, the SRC value, the NormI value, the PASI value, the PODI value, the HRMDI value, the HZDI value, and parameters derived from these values) from the acquired signals, the preprocessed signals and/or the features; and/or (iv) assess risk and/or severity of a cardiovascular disease in the subject with the cardiovascular-related physiological parameters and/or the vascular metric space. The processor can be used to detect arterial damage and risk of cardiovascular disorders using a threshold judgment. The assessment results generated from the processor can be transmitted via Bluetooth to end users and clients for accurate and reliable decision making.

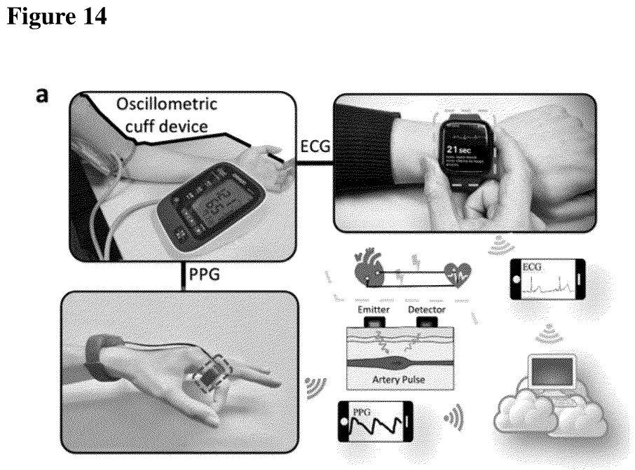

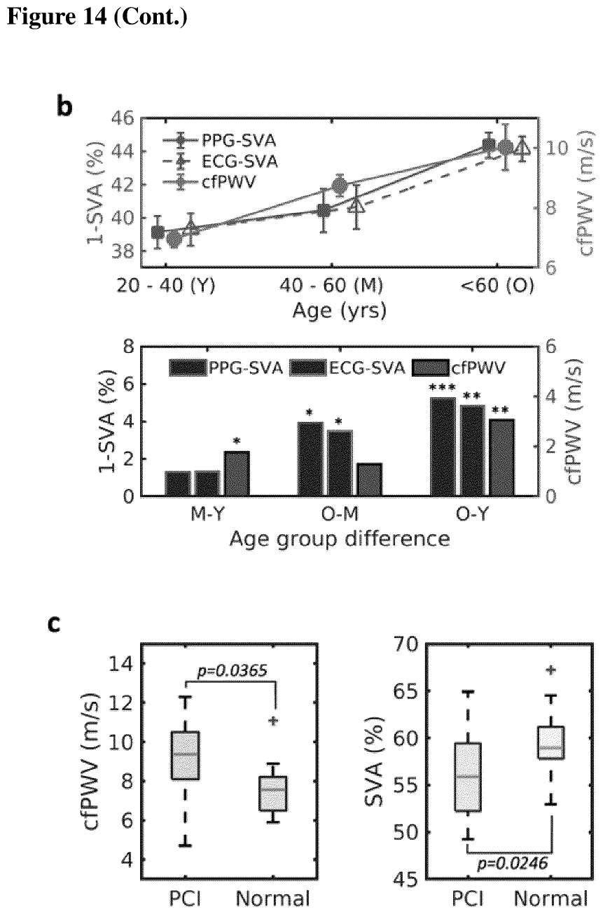

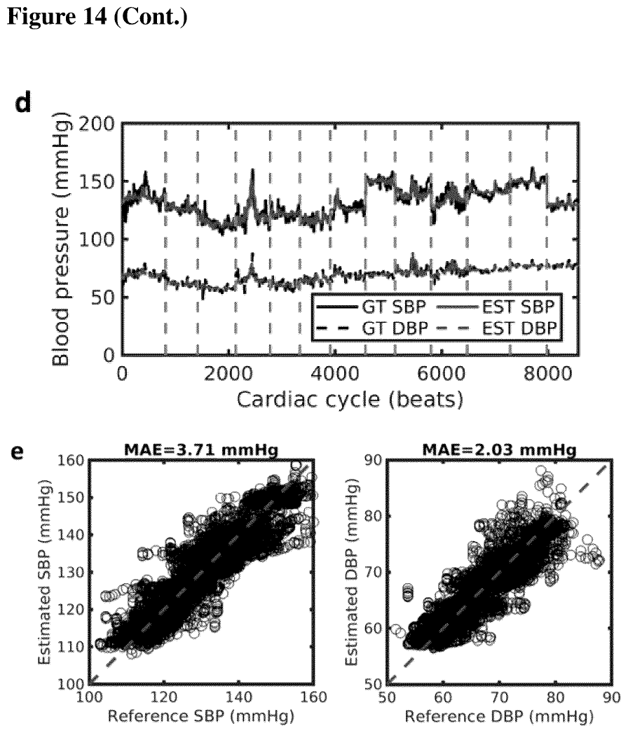

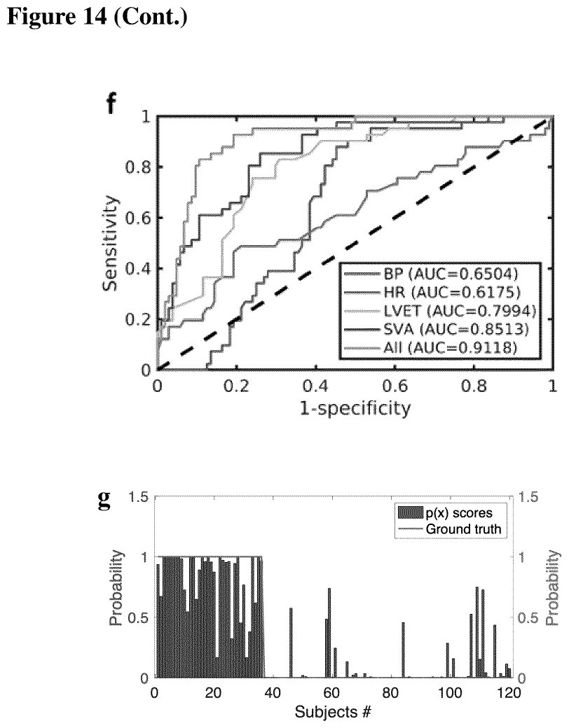

As compared with traditional systems used in cardiac monitoring that uses physiological data such as blood pressure (BP), heart rate (HR), left ventricular ejection time (LVET) or the like, the system in the present application enables more accurate assessment of risk and/or severity of a cardiovascular disease in a subject, as shown in e.g., FIG. 14(f).

Further, the system in the present application can be implemented as a system that has new functions, and/or has an improved performance as compared to traditional systems.

For example, the system in the present application enables the evaluation of arterial stiffness (AS), which cannot be performed by traditional wearable devices. The conventional method of AS measurement, i.e., the carotid-femoral pulse wave velocity (cfPWV) test, cannot be performed by wearable and flexible devices, since the cfPWV test must be performed by a medical professional on a patient lying supine in a quiet environment. In contrast, as described herein, the system in the present application can be used to assess arterial stiffness with the Ratio_{SVA} value derived SVA values.

For another example, the system in the present application enables continuous tracking of blood pressure. Traditional wearable devices that are based on pulse transit time (PTT) and data-driven BP measurement methods can achieve continuous cuffless monitoring, but these wearable devices cannot precisely interpret BP-related hemodynamics from the crosstalk between macrocirculation and microcirculation, which may result in their performance decreasing over time. By contrast, the system in the present application directly calculates the dynamic changes in blood volume distribution between the elastic arteries and peripheral arterioles during each heartbeat cycle, and facilitates the tracking of changes in BP.

Further, unlike the traditional systems used in cardiac monitoring, the system in the present application allows for the assessment of cardiac, vascular, and/or organ injury risk during daily monitoring.

In software development, the realization of the algorithm in the present application is also more practicable on smart wearable devices compared with other sophisticated methods.

Some embodiments of the present application present models and measurement protocols for stroke-volume-allocation (SVA) and systemic reflection coefficient (SRC), quantifying arterial cushioning function and evaluating excess energy-induced organ damage, respectively. In some embodiments, the present application aims to extend the capabilities of wearable sensors or non-contact cardiac pulse sensors to enable arterial system risk assessment, thus enabling (a) pulse wave-based scenario adaptive arterial stiffness (AS) assessment, (b) scenario adaptive ambulatory cuffless blood pressure tracking, (c) cardiovascular disease-related heart damage (CHD) assessment, and (d) assessment of vital organ damage, including those in the heart, brain, and kidneys. Specifically, cerebrovascular diseases such as mild cognitive impairment and vascular dementia can be detected by the methods and systems of the present application. In some embodiments, to make off-the-shelf sensors workable in SVA and SRC measurements, an iterative measurement protocol for extending the functionality of existing cardiac sensors is provided in the present application. This iterative measurement protocol, combined with cardiac sensors and snapshot blood pressure (BP) measurement, can provide low-cost, real-time, long-term, unobtrusive, and continuous arterial system risk assessment and allow contact and/or contactless cardiovascular disease monitoring. In some embodiments, cardiovascular parameters and functions may be performed by the system in the present application in everyday scenarios such as resting, exercising, eating, and sleeping, etc. In some embodiments, the system in the present application can complete arterial stiffness measurements in less than 30 seconds, much faster than conventional carotid-femoral pulse wave velocity (cfPWV) measurements.

Provided herein are methods, devices and systems for assessing risk and/or severity of a cardiovascular disease in a subject. In some embodiment, the methods, devices, and systems can provide risk alerts, visualizations, and treatment recommendations based on input core vascular indices, such as pharmacy-related drug sales and location and time of appointments for near-by hospital visits, by a pre-trained large language model (LLM), see e.g., FIG. 2. The methods and systems of the present application may utilize a plurality of SVA values and/or one or more parameters derivable from the SVA values to assess the cardiovascular disease, e.g., for its risk and/or severity. In some embodiments, an SVA value represents a ratio of a stroke volume stored in the aorta of the subject during the systolic phase in a cardiac cycle to a total stroke volume pumped from the heart of the subject during the cardiac cycle. In some embodiments, an SVA value is equal to a ratio of a stroke volume stored in the aorta of the subject during the systolic phase in a cardiac cycle to a total stroke volume pumped from the heart of the subject during the cardiac cycle. In some embodiments, an SVA value is calculated from a blood pressure waveform. For example, from the blood pressure waveform, area under the blood pressure waveform during diastolic phase of the cardiac cycle (defined as A_{d}) and area under the blood pressure waveform during systolic phase of the cardiac cycle (defined as Aₛ) can be obtained, and an SVA value can represent (in some particular cases, is equal to) the ratio of A_{d} to the sum of A_{d} and As. SVA can be used to quantify arterial cushioning function in a subject.

The one or more parameters derivable from the SVA include but are not limited to systemic reflection coefficient (SRC). An SRC value can be used to evaluate organ damage induced by excess pulsatile energy. An SRC value can be calculated from an SVA value as detailed hereinafter.

As used herein, a “normal subject”, a “normal individual”, “a healthy subject” and “a healthy individual” are used interchangeably and refer to an individual without a cardiovascular disease, and can be a fully healthy individual, unless indicated otherwise. In some embodiments, a value derived from a population of healthy individuals is used as a reference value for comparison with a test value measured for a test subject. The healthy individuals and the test subject may have common demographic features. For example, they can have the same gender, and/or are within a same age group (e.g., people of 30-40 years old, or 40-50 years old).

In a first aspect, provided is a system (e.g., a medical system or a wearable device (e.g., a smart watch)) for assessing risk and/or severity of a cardiovascular disease in a subject, comprising:
a blood pressure measuring device and/or a cardiac rhythm monitor, wherein the blood pressure measuring device is configured to perform a blood pressure measurement on the subject, and the cardiac rhythm monitor is configured to perform a cardiac rhythm measurement on the subject;
a processor configured to (i) determine, from data from the blood pressure measurement and/or the cardiac rhythm measurement in the subject, a plurality of stroke volume allocation (SVA) values, (ii) calculate an average SVA value from the plurality of SVA values and compare the average SVA value with a reference SVA value derived from a population of healthy individuals, and (iii) for an average SVA value which is below the reference SVA value, generate an assessment result indicating that the subject has an increased risk and/or severity of the cardiovascular disease as compared with the healthy individuals; and
an output device (e.g. a display) for outputting the assessment result;
wherein an SVA value represents a ratio of a stroke volume stored in the aorta of the subject during the systole phase in a cardiac cycle to a total stroke volume pumped from the heart of the subject during the cardiac cycle.

In some embodiments of the first aspect, an SVA value is a ratio of a stroke volume stored in the aorta of the subject during the systole phase in a cardiac cycle to a total stroke volume pumped from the heart of the subject during the cardiac cycle.

In some embodiments of the first aspect, the data is from a blood pressure measurement and comprises a blood pressure waveform in the cardiac cycle. In some particular embodiments, an SVA value may be generated by:
i) calculating the area under the blood pressure waveform during the diastolic phase of the cardiac cycle, defined as A_{d};
ii) calculating the area under the blood pressure waveform during the systolic phase of the cardiac cycle, defined as Aₛ; and
iii) calculating the SVA value by the following equation:

$$S V A = \frac{Ad}{As + Ad} .$$

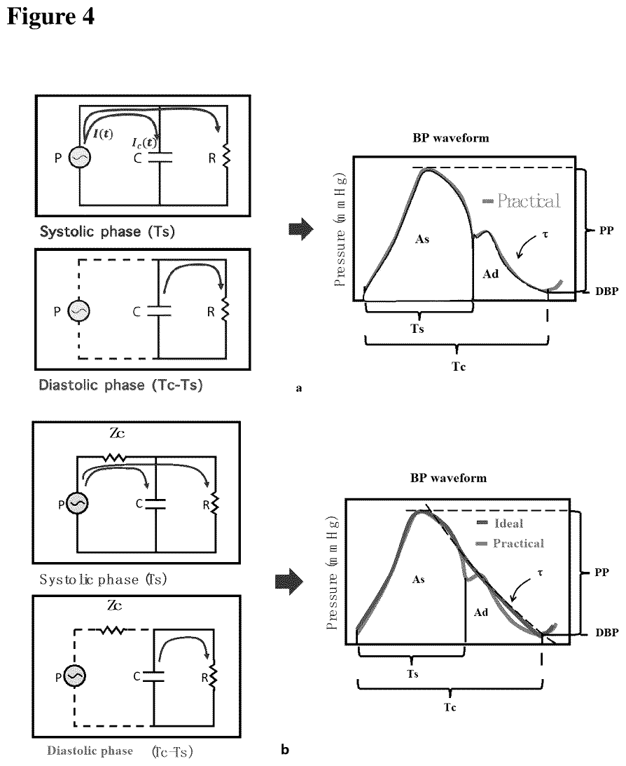

In some embodiments, the blood pressure waveform comprises a first valley point, a dicrotic notch point and a second valley point. As shown in FIG. 4, the systolic phase is a phase substantially between the first valley point and the dicrotic notch point, and the diastolic phase may be a phase substantially between the dicrotic notch point and the second valley point. In some embodiments, Aₛ is obtained by calculating the area under the blood pressure waveform from the first valley point to the dicrotic notch point. In some embodiments, A_{d} is obtained by calculating the area under the blood pressure waveform from the dicrotic notch point to the second valley point.

In some embodiments of the first aspect, the data is from a blood pressure measurement and a cardiac rhythm measurement, and comprises a blood pressure waveform and a pulse waveform in the cardiac cycle. In some particular embodiments, an SVA value is generated by: a) extracting a diastolic blood pressure (DBP) and a mean blood pressure (MBP) from the data, and extracting a cardiac duration and a systolic duration from the pulse waveform; and b) calculating the SVA value by the following equation:

$$S V A {=}^{Δ} ( δ + Δ {δ}_{pulse} ) \cdot ( 1 - \frac{Ts}{Tc} ) ,$$

wherein Tₛ is the systolic duration, T_{c} is the cardiac duration,

$$δ = \frac{D B P + M B P}{2 \cdot D B P}$$

and Δδₚᵤₗₛₑ is a compensation term associated with status of an artery in the subject. In some particular embodiments, Δδₚᵤₗₛₑ is expressed as the relative change in a pulse waveform measured over one cardiac cycle while the subject is at the test status to a pulse waveform measured when the subject is at a resting status. For example,

$$Δ {δ}_{pulse} = \frac{{Ad}_{testing} - {Ad}_{resting}}{{Ad}_{resting}} .$$

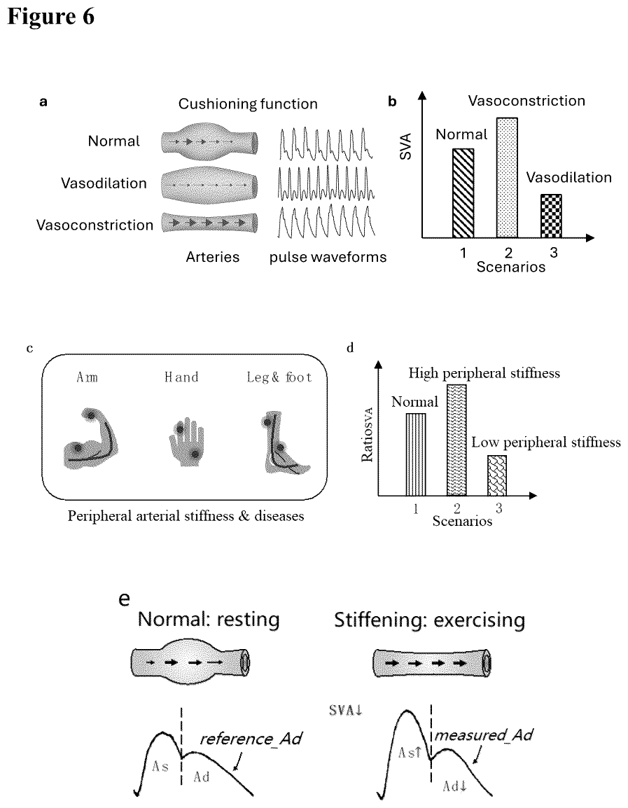

The Ad_{testing} is the area under a pulse waveform during the diastolic phase of the cardiac cycle when the subject is at a test status, and the Ad_{resting} is the area under a pulse waveform during the diastolic phase of a cardiac cycle when the subject is at a resting status. FIG. 6 (e) shows that when the status of the subject shifts from the resting status to the exercising status, Ad changes with the pulse, resulting in a change (e.g., a decrease) in the SVA. The compensation term Δδₚᵤₗₛₑ can be used to obtain precise scenario adaptive SVA values.

In some particular embodiments, the test status is selected from the group consisting of a resting status, a status of vasodilation, a status of vasoconstriction and any combination thereof. A person skilled in the art commonly knows that arterial conditions of an individual change dynamically with time. As shown in FIG. 6 (a, b and e), different arterial conditions correspond to different values of the measured_Ad (i.e., the Ad_{testing}) and SVA.

Information from a blood pressure measurement and/or information from a cardiac rhythm measurement, e.g., blood pressure (BP), heart rate (HR), left ventricular ejection time (LVET) and so on, has been be used to assess risk and/or severity of cardiovascular diseases. In the present application, the information is used as raw data to calculate SVAs, and the SVAs are then used to support comprehensive cardiovascular and cerebrovascular disease assessment. As shown in FIG. 14 (f), the SVAs show higher relevance in the classification of arterial system than other indicators, including BP, HR and LVET. Therefore, one advantage of the present application is that the SVA is a more accurate indicator for assessing risk and/or severity of cardiovascular diseases. The methods of the present application and systems (e.g., wearable devices) for implementing the methods can more accurately assess the risk and/or severity of cardiovascular diseases than conventional systems or conventional wearable devices.

In some embodiments of the first aspect, the processor is further configured to:
(1) by using the steps a) and b) above, generate a plurality of pairs of blood pressure (BP) and SVA values and calculating an average δ at a plurality of first timepoints, wherein a single blood pressure waveform and a single pulse waveform obtained at the same timepoint are used to determine each pair of BP and SVA values,
(2) determine bₛ*, kₛ*, PSR and b by feeding the plurality of pairs of BP and SVA values into a formula for least square method to minimize an error in BP as follows:

$${arg min}_{{b}_{s}^{*} , b , PSR , {k}_{s}^{*}} ‖ BP - \frac{{b}_{s}^{*} - S V A \cdot b}{S V A \cdot ( PSR - 1 ) + 1 - {k}_{s}^{*}} ‖ ,$$

(3) generate SVA values at a plurality of second timepoints from a pulse waveform in the cardiac cycle by the following equation:

$$SVA {=}^{△} ( δ + Δ {δ}_{pulse} ) \cdot ( 1 - \frac{Ts}{Tc} ) ,$$

wherein Tₛ, T_{c}, and Δδₚᵤₗₛₑ are defined above and δ is the average δ calculated in step (1); and (4) calculate BP values through the SVA values at the plurality of second timepoints according to the following equation:

$$BP = \frac{{b}_{s}^{*} - SVA \cdot b}{SVA ( PSR - 1 ) + 1 - {k}_{s}^{*}} .$$

The least square method is well known in the art as a statistical technique to determine the best fit line for a set of data points, and can be used to determine the coefficients bₛ*, kₛ*, PSR and b.

In some particular embodiments, the system of the present application has a low power consumption, since the computational complexity in signal processing is low. Consequently, the system in the present application can beneficially support continuous tracking of blood pressure by SVA values. SVA can be determined by any one of related method described herein, for example by the following equation:

$$SVA {=}^{△} ( δ + Δ {δ}_{pulse} ) \cdot ( 1 - \frac{Ts}{Tc} ) .$$

In some embodiments of the first aspect, the processor is further configured to determine a probability of the cardiovascular disease by the following equation:

p(x)=1−1/(1+e^{-(β}^{₀}^{+β}^{₁}^{x}^{₁}^{+β}^{₂}^{x}^{₂}^{+β}^{₃}^{x}^{₃}^{+β}^{₄}^{x}^{_{4+β}}^{₅}^{x}^{₅}⁾),

wherein X₁ is a heart rate (HR), X₂ is a left ventricular ejection time (LVET), X₃ is a systolic blood pressure (SBP), X₄ is a diastolic blood pressure (DBP), X₅ is a SVA value, β₀=−92.7477, β₁=0.3109, β₂=41.5207, β₃=0.0627, β₄=−0.1047, β₅=0.9826. The value of p(x) is from 0 to 1. An increase in the p(x) value in the subject as compared with a reference p(x) value derived from a population of healthy individuals may indicate that the subject has a risk of developing the cardiovascular disease. In some embodiments, the reference p(x) value is below 0.5, such as 0.4, 0.3, 0.2, 0.1 or 0. In some embodiments, an individual with a p(x) value of 0.5 is determined to have a risk of developing the cardiovascular disease. In some embodiments, an individual with a p(x) value of above 0.5 is determined to have a risk of developing the cardiovascular disease. In some embodiments, an individual with a p(x) value of above 0.6 is determined to have a risk of developing the cardiovascular disease. In some embodiments, an individual with a p(x) value of above 0.7 is determined to have a risk of developing the cardiovascular disease. In some embodiments, an individual with a p(x) value of above 0.8 is determined to have a risk of developing the cardiovascular disease. In some embodiments, an individual with a p(x) value of above 0.9 is determined to have a risk of developing the cardiovascular disease.

In some embodiments of the first aspect, the blood pressure measurement and/or the cardiac rhythm measurement are performed on a peripheral artery and a central artery, respectively. In some particular embodiments, the processor is further configured to:
i) determine, from data from the blood pressure measurement and/or the cardiac rhythm measurement in the subject, a first SVA value for the peripheral artery and a second SVA value for the central artery;
ii) determine RatioSVA as a ratio of the first SVA value to the second SVA value, and
iii) assess arterial stiffness with the RatioSVA.

An increase in the RatioSVA value in the subject as compared with a reference RatiosVA value derived from a population of healthy individuals may indicate a higher degree of arterial stiffness in the peripheral artery of the subject than the population of healthy individuals.

In some embodiments of the first aspect, the data is from a blood pressure measurement and a cardiac rhythm measurement, and the processor is further configured to assess an organ damage induced by a pulsatile energy in the cardiovascular system of the subject with a systemic reflection coefficient (SRC) value calculated by the following equation:

$$SRC = 2 \frac{SVA}{\frac{( {e}^{\frac{{T}_{c} - {T}_{s}}{τ}} - 1 ) \cdot DBP}{PP}} - 1 ,$$

wherein DBP is a diastolic blood pressure, PP is a pulse pressure, Tₛ is a systolic duration from a pulse waveform, T_{c} is a cardiac duration from the pulse waveform, and wherein DBP and PP are from the blood pressure measurement, Ts and Tc are from the cardiac rhythm measurement, and τ is calculated by an implicit solution, e.g., by the following equation:

$$\frac{DBP}{PP} = \frac{{e}^{\frac{- {T}_{s}}{τ}} \cdot \frac{1}{{( \frac{1}{τ} )}^{2} + {( \frac{π}{{T}_{s}} )}^{2}} [ ( {e}^{\frac{{T}_{s}}{τ}} + 1 ) {( \frac{π}{{T}_{s}} )}^{2} ]}{2 \cdot ( {e}^{\frac{{T}_{c} - {T}_{s}}{τ}} - {e}^{\frac{- {T}_{s}}{τ}} )} .$$

An increase in the SRC value in the subject as compared with a reference SRC value derived from a population of healthy individuals may indicate that an organ damage in the subject.

In some particular embodiments, the processor is further configured to assess the cardiovascular disease with indicators derived from the SRC, the SVA, a mean blood pressure (MBP), and the PP as follows:

NormI= [symbol] · [symbol] / [symbol] ;

PASI= [symbol] / [symbol] ;

PODI= [symbol] / [symbol] · [symbol] ;

HRMDI= [symbol] · [symbol] · [symbol] .

HZDI=1/( [symbol] · [symbol] );

wherein the symbol ~ for each parameter represents a ratio of a value measured for the subject to a reference value derived from a population of healthy individuals.

In some embodiments, the reference value is derived from a population of healthy individuals. For example, the reference value may be an average, a median, a mode, a quartile (e.g., a first quartile, a second quartile, or a third quartile) of data points from the population of healthy individuals. In some particular embodiments, a population of healthy individuals may have an average blood pressure of about 100 mmHg, an average heart rate of about 60 Beats per Minute (BPM), an average systolic duration of about 0.3 s, an average SVA of about 56%, an average SRC of about 0.8, etc. In some embodiments of the determination of the indicator NormI, MBP, SVA and SRC values are measured for the subject, and each of them is then normalized to (e.g., divided by) reference MBP, SVA, SRC values for the population of healthy individuals, respectively, and the normalized values are used to determine the indicator NormI. Other indicators, including PASI, PODI, HRMDI and HZDI can be determined in a similar way.

In some particular embodiments, the population of healthy individuals is within the same age group and/or the same gender group as the subject.

In some embodiments, an increase in NormI in the subject from a first timepoint to a second timepoint indicates an improvement in the cardiovascular system of the subject from a first timepoint to a second timepoint.

In some embodiments, an increase in PASI in the subject from a first timepoint to a second timepoint indicates a higher risk for arterial stiffness due to poor cushioning function and a high SRC in the subject at the first timepoint than that at the second timepoint.

In some embodiments, an increase in PODI in the subject from a first timepoint to a second timepoint indicates a higher risk for pulsatile-induced organ damage due to poor pulsation reflexes, poor cushioning function, and a large pulsation component of BP in the subject at the first timepoint than that at the second timepoint.

In some embodiments, an increase in HRMDI in the subject from a first timepoint to a second timepoint indicates a higher risk for systemic-vascular-resistance-induced microcirculation damage in the subject at the first timepoint than that at the second timepoint.

In some embodiments, an increase in HZDI in the subject from a first timepoint to a second timepoint indicates a higher risk for hazardous cardiovascular system with arterial stiffness and pulsatile-induced organ damage in the subject at the first timepoint than that at the second timepoint.

The cardiovascular disease may be selected from the group consisting of mild cognitive impairment, stroke, dementia, valvular aortic stenosis, aortic incompetence and any combination thereof. In some embodiments, the cardiovascular disease may be a condition resulting from mild cognitive impairment, stroke, dementia, valvular aortic stenosis, aortic incompetence and any combination thereof.

In some embodiments of the first aspect, the assessment result comprises an alert about the risk and/or severity of the cardiovascular disease, and/or a treatment recommendation.

In some embodiments of the first aspect, the system further comprises a memory configured to store data from the blood pressure measurement and/or the cardiac rhythm measurement, the reference SVA value and/or the assessment result.

In some embodiments of the first aspect, the blood pressure measuring device is selected from the group consisting of a pressure sensor, a digital sphygmomanometer, stethoscope sphygmomanometer and any combination thereof.

In some embodiments of the first aspect, the cardiac rhythm monitor is selected from the group consisting of a cardiac rhythm monitor based on electrocardiogram (ECG), a cardiac rhythm monitor based on photoplethysmography (PPG), a cardiac rhythm monitor based on phonocardiography (PCG), a cardiac rhythm monitor based on impedance cardiography (ICG), a cardiac rhythm monitor based on ultrasound, a cardiac rhythm monitor based on ballistocardiography (BCG), a cardiac rhythm monitor based on seismocardiographic (SCG) and any combination thereof.

In some embodiments of the first aspect, the blood pressure measuring device and the cardiac rhythm monitor are configured to make simultaneous measurements.

Also provided herein is a system for physiological parameter monitoring in a subject, comprising:
at least one sensor for acquiring a physiological data; and
a processor communicatively coupled to sensor configured to perform the steps of (a) preprocessing physiological data to reduce noise; (b) applying a feature extraction method for generating optimized physiological parameters; and an output module for outputting optimized physiological parameters.

The system can provide an enhanced performance in the identification of cardiovascular disease risk and/or severity through synergistic operation of the processor and the sensor, as compared with traditional systems used in cardiac monitoring.

Also provided herein is a system for quantitative monitoring of multiple cardiovascular parameters, comprising:
at least one sensor for acquiring a physiological data; and
a processor communicatively coupled to sensor configured to perform the steps of (a) preprocessing physiological data to reduce noise; (b) applying a feature extraction algorithm for generating optimized physiological parameters; (c) performing multiple cardiovascular parameters calculation and cardiovascular disease risk and/or severity identification; and an output module for outputting optimized physiological parameters.

The system can have improved efficiency of cardiovascular disease risk and/or severity identification through the synergistic operation of the processor and the sensor, as compared with traditional systems used in cardiac monitoring.

The system described above may be implemented by a computer system. In some embodiments, the system described above may be implemented by a wearable device.

In some embodiments of the first aspect, the system enables device-based dynamic management of the cardiovascular disease by providing the subject with strategies to effectively reduce the risk of cardiovascular disease based on the assessed risk and/or severity of the cardiovascular disease. In some particular embodiments, a first set of values for hemodynamic parameters in the subject with the cardiovascular disease, including SVA and SRC and the corresponding NormI, PASI, PODI, HRMDI, and HZDI, etc. is determined before a therapy such as breathing exercise and/or drug treatment for the subject; after the therapy, a second set of values for the hemodynamic parameters is determined; and the first set of values is compared with the second set of values, to determine whether the treatment is effective and/or to provide feedback for adjusting the therapy until the values for hemodynamic parameters achieve to normal values. In particular, the first set of values and/or the second set of values may be compared with standardized reference values (e.g., values measured for a healthy individual), to evaluate the condition of the subject.

In some embodiments of the first aspect, the blood pressure measuring device (e.g., a pressure sensor) is used to measure the blood pressure in the subject, and is able to provide a blood pressure waveform in a cardiac cycle of the subject. In some embodiments of the first aspect, the cardiac rhythm monitor (e.g., a pulse sensor) is used to monitor the cardiac rhythm in the subject, and is able to provide a pulse waveform in the cardiac cycle of the subject. The blood pressure measuring device and the cardiac rhythm monitor may be configured to make simultaneous or sequential measurements.

The blood pressure measuring device may be invasive or noninvasive. The blood pressure measuring device may be configured to measure blood pressure continuously or intermittently.

The cardiac rhythm monitor may be selected from the group consisting of an electrical device, a mechanical device, an optical device, an electromagnetic device and any combination thereof. The cardiac rhythm monitor may measure cardiac rhythm in a mode which is contact or contactless. In some embodiments, the mode may be real-time, continuous, wearable and/or portable. The cardiac rhythm monitor may be a wearable cardiac rhythm monitor, e.g., a smartwatch, fitness trackers, a smart glass, a smart ring, a smart clothing, smart earbuds, a smart shoe, a smart jewelry, and a non-contact sensor.

In a second aspect, provided is a method for assessing risk and/or severity of a cardiovascular disease in a subject, comprising:
generating data from a blood pressure measurement and/or a cardiac rhythm measurement in the subject;
determining from the data a plurality of stroke volume allocation (SVA) values;
calculating an average SVA value from the plurality of SVA values and comparing the average SVA value with a reference SVA value derived from a population of healthy individuals;
for an average SVA value which is below the reference SVA value, generating an assessment result indicating that the subject has an increased risk and/or severity of the cardiovascular disease as compared with the healthy individuals; and
outputting the assessment result;
wherein an SVA value represents a ratio of a stroke volume stored in the aorta of the subject during the systole phase in a cardiac cycle to a total stroke volume pumped from the heart of the subject during the cardiac cycle.

In a third aspect, provided is a computer-readable storage medium having stored therein program code instructions that, when executed by a processor in a computer system, cause the processor to perform a method in the second aspect.

A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. Computer readable media may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

In a fourth aspect, provided is a computer product comprising a non-transitory computer readable medium storing a plurality of instructions that when executed cause a computer system to perform a method in the second aspect.

In a fifth aspect, provided is a system comprising the computer product described in the fourth aspect and one or more processors for executing instructions stored on the computer readable medium.

In a sixth aspect, provided is a system comprising means for performing a method in the second aspect.

In a seventh aspect, provided is a system comprising one or more processors configured to perform a method in the second aspect.

In some embodiments, the system described herein may be implemented as a wearable device, a desktop computer, laptop computer, tablet computer, mobile device (e.g., smart phone), or the like. Such system may include one or more processors to execute program code (e.g., general purpose microprocessors usable as a central processing unit (CPU) and/or special purpose processors such as graphics processors (GPUs) that may provide enhanced parallel processing capability); memory and other storage devices to store program code and data; user input devices (e.g., keyboards, pointing devices such as a mouse or touchpad, microphones); user output devices (e.g., display devices, speakers, printers); combined input/output devices (e.g., touchscreen displays); signal input/output ports; network communication interfaces (e.g., wired network interfaces such as Ethernet interfaces and/or wireless network communication interfaces such as Wi Fi); and so on.

Computer programs incorporating features of the present application that can be implemented using program code may be encoded and stored on various computer readable storage media. Suitable media include magnetic disk or tape, optical storage media such as compact disk (CD) or DVD (digital versatile disk), flash memory, and other non-transitory media. Computer readable media encoded with the program code may include an internal storage medium of a compatible electronic device and/or external storage media readable by the electronic device that can execute the code. In some instances, program code can be supplied to the electronic device via Internet download or other transmission paths.

A system of the present application can include a plurality of the same components or subsystems, e.g., connected together by an external interface, by an internal interface, or via removable storage devices that can be connected and removed from one component to another component. In some embodiments, systems, wearable devices, or apparatuses can communicate over a network.

Some aspects of the present application can be implemented in the form of control logic using hardware circuitry (e.g. an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Also provided herein is a system for the assessing risk and/or severity of a cardiovascular disease in a subject comprises:
a blood pressure measuring device (e.g., a pressure sensor) configured to perform a blood pressure measurement on the subject; and
a processor configured to determine, from data from the blood pressure measurement, a plurality of stroke volume allocation (SVA) values, and assess risk and/or severity of the cardiovascular disease based on the SVA values,
wherein an SVA value represents a ratio of a stroke volume stored in the aorta of the subject during the systole phase in a cardiac cycle to a total stroke volume pumped from the heart of the subject during the cardiac cycle.

Also provided is a method for using the system to assess risk and/or severity of a cardiovascular disease in a subject, comprising:
performing a blood pressure measurement on the subject with a blood pressure measuring device (e.g., a pressure sensor); and
by the processor, determining from data from the blood pressure measurement, a plurality of stroke volume allocation (SVA) values, and assessing risk and/or severity of the cardiovascular disease based on the SVA values;
wherein an SVA value represents a ratio of a stroke volume stored in the aorta of the subject during the systole phase in a cardiac cycle to a total stroke volume pumped from the heart of the subject during the cardiac cycle.

Also provided is a system for the assessing risk and/or severity of a cardiovascular disease in a subject comprises:
a blood pressure measuring device (e.g., a pressure sensor) configured to perform a blood pressure measurement on the subject;
a cardiac rhythm monitor (e.g., a pulse sensor) configured to perform a cardiac rhythm measurement on the subject; and
a processor configured to determine, from data from the blood pressure measurement and the cardiac rhythm measurement in the subject, a plurality of stroke volume allocation (SVA) values, and assess risk and/or severity of the cardiovascular disease based on the SVA values,
wherein an SVA value represents a ratio of a stroke volume stored in the aorta of the subject during the systole phase in a cardiac cycle to a total stroke volume pumped from the heart of the subject during the cardiac cycle.

Also provided is a method for using the system to assess risk and/or severity of a cardiovascular disease in a subject, comprising:
performing a blood pressure measurement on the subject with a blood pressure measuring device (e.g., a pressure sensor);
performing a cardiac rhythm measurement on the subject with a cardiac rhythm monitor (e.g., a pulse sensor); and
by the processor, determining from data from the blood pressure measurement and the cardiac rhythm measurement, a plurality of stroke volume allocation (SVA) values, and assessing risk and/or severity of the cardiovascular disease based on the SVA values;
wherein an SVA value represents a ratio of a stroke volume stored in the aorta of the subject during the systole phase in a cardiac cycle to a total stroke volume pumped from the heart of the subject during the cardiac cycle.

The blood pressure measuring device and the cardiac rhythm monitor may be configured to make simultaneous or sequential measurements.

In some embodiments, the system comprises an input device configured to receive an input, e.g., from a user, a manufacturer and the like. The input can be, but is not limited to, the subject's medical information (e.g., medical history, activity levels, etc.), the subject's physiological data (e.g., blood pressure waveforms, pulse waveforms, and parameters relating to blood pressure or cardiac rhythm such as diastolic blood pressure (DBP), a mean blood pressure (MBP), systolic duration (Tₛ), cardiac duration (Tc), heart rate (HR), left ventricular ejection time (LVET), etc.), the subject's status (e.g., a resting status, a status of vasodilation, or a status of vasoconstriction), and other information that can be involved in the assessment of risk and/or severity of a cardiovascular disease in a subject. The input device may be selected from the group consisting of a touchscreen, a microphone, a light pen, a keyboard, a scanner, a webcam, and another input device known in the art.

For example, the system may comprise the input device, a cardiac rhythm monitor (e.g., a pulse sensor) configured to perform a cardiac rhythm measurement on the subject, and a processor.

Also provided is a method for using the system to assess risk and/or severity of a cardiovascular disease in a subject, comprising:
receiving, from the input device, data from a blood pressure measurement in the subject;
performing a cardiac rhythm measurement on the subject with the cardiac rhythm monitor (e.g., a pulse sensor); and
by the processor, determining from data from the blood pressure measurement and the cardiac rhythm measurement, a plurality of stroke volume allocation (SVA) values, and assessing risk and/or severity of the cardiovascular disease based on the SVA values;
wherein an SVA value represents a ratio of a stroke volume stored in the aorta of the subject during the systole phase in a cardiac cycle to a total stroke volume pumped from the heart of the subject during the cardiac cycle.

In some embodiments, the system comprises a memory configured for storing the data from the blood pressure measurement and/or the cardiac rhythm measurement. The memory may be coupled to the processor.

For example, the system may comprise a memory configured for storing the data from the blood pressure measurement, a cardiac rhythm monitor (e.g., a pulse sensor), and a processor.

Also provided is a method for using the system to assess risk and/or severity of a cardiovascular disease in a subject, comprising:
receiving, from the memory, data from a blood pressure measurement in the subject;
performing a cardiac rhythm measurement on the subject with the cardiac rhythm monitor (e.g., a pulse sensor); and
by the processor, determining from data from the blood pressure measurement and the cardiac rhythm measurement, a plurality of stroke volume allocation (SVA) values, and assessing risk and/or severity of the cardiovascular disease based on the SVA values;
wherein an SVA value represents a ratio of a stroke volume stored in the aorta of the subject during the systole phase in a cardiac cycle to a total stroke volume pumped from the heart of the subject during the cardiac cycle.

In some embodiments, the system comprises an output device (e.g., a display, a speaker, etc.) for outputting one or more of the SVA values, the RatioSVA value, the BP value calculated with the SVA values, the p(x) value, the SRC value, the NormI value, the PASI value, the PODI value, the HRMDI value, the HZDI value, and parameters derived from these values.

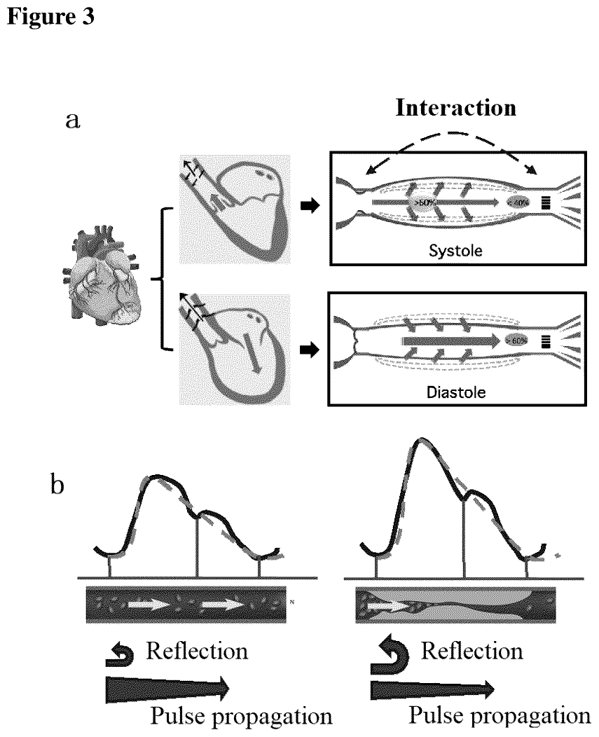

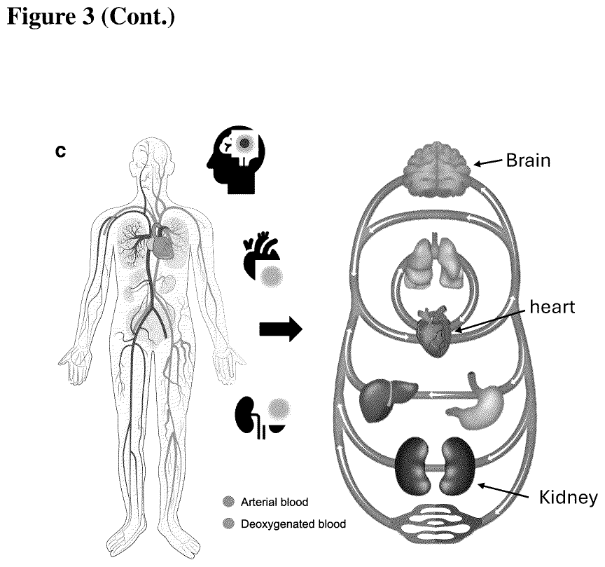

FIG. 3 presents the concepts of arterial cushioning function and the pulsatile energy-induced organ damage in the human cardiovascular system according to some embodiments of the present application. The arterial cushioning function, described by SVA, plays an important role in buffering blood volume and then supporting continuous volume or energy to distal arteries and organs during the cardiac cycle (FIG. 3 (a)). The excess pulsatile energy, described by SRC, generates from the heart, then propagates to target organs that may cause potential organ damage in the circulation (FIG. 3 (b & c)). In some embodiments, the present application provides a quantitative model to allow for extraction of SVA and SRC from the electrical signals obtained by wearable sensors and hence support comprehensive cardiovascular and cerebrovascular disease assessment.

FIG. 4 illustrates an exemplary working principle of the SVA and SRC models which associates with the specific Windkessel models according to some embodiments of the present application.

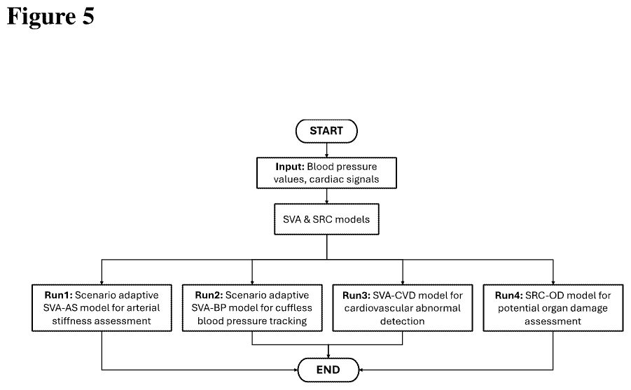

FIG. 5 shows an exemplary system workflow of SVA and SRC measurement in cardiovascular system risk assessment, including arterial stiffness assessment, blood pressure tracking, and CHD abnormality detection, and potential vital organ damage assessment, according to some embodiments. To be specific, the SVA and SRC measurement may require blood pressure recording and a cardiac signal as input, as shown in FIG. 5. The cardiac signal may be generated by any type of a cardiac sensor that measures ambulatory heart rhythm, which includes at least systolic and cardiac cycles. The recorded blood pressure (BP) can be continuous or intermittent. The continuous and intermittent snapshot BP values can be observed from arterial segments, such as arm, carotid, finger, and leg. Besides, the wearable cardiac devices can be off-the-shelf sensors, such as those based on ECG, PPG, PCG, impedance cardiography (ICG), ultrasound, pressure, ballistocardiography (BCG), and seismocardiography (SCG) etc., which can be attached on any part of the body accordingly. Thus, the methods and systems of the present application facilitate family-centered self-management of cardiovascular care and hospital-centered prognosis of disease and early medical intervention.

In some embodiments, the SVA and SRC models may enable four basic CVD monitoring related functions that can be conducted by cardiac sensors and a BP measurement machine, including: (1) pulse wave-based scenario adaptive stroke-volume-allocation versus arterial stiffness (SVA-AS) model for arterial stiffness assessment, (2) scenario adaptive stroke-volume-allocation versus blood pressure (SVA-BP) model for continuous BP tracking, (3) stroke-volume-allocation versus cardiovascular disease (SVA-CVD) model for CHD abnormality assessment, and (4) systemic reflection coefficient versus organ damage (SRC-OD) model for vital organ damage assessment including the heart, brain and kidney, etc. Some aspects of the techniques for SVA and SRC measurements are detailed in the following sections, including modeling and working principle, validation, sensing technology, and application in wearable devices.

### Exemplary Modeling and Working Principles

Some exemplary modeling and working principles of the present application are provided hereinafter, including those in Run1 (Scenario adaptive SVA-AS model), Run2 (SVA-BP model), Run3 (SVA-CVD model) and Run4 (SRC-OD model).

As shown in FIG. 4, the SVA model may be used to describe the cushioning function of an artery, which buffers the energy from heart and efficiently support blood volume to target organs in each heartbeat cycle. In some embodiments, the stroke volume allocation (SVA) can be defined as the ratio of the stroke volume in the arterial compliance during the systole phase to the total stroke volume pumped from the heart during the same cardiac cycle, as shown in Equation (1):

$$SVA = \frac{{SV}_{c}}{SV} = \frac{Ad}{As + Ad} ,$$

wherein SVc is the stored volume of the aorta during the systolic duration; SV is the stroke volume during each heartbeat; A_{d} is the diastolic phase area of the blood pressure; Aₛ is the systolic phase area of the blood pressure. Hence, in some embodiments, the SVA can be expressed as the area ratio of diastolic BP waveform (A_{d} in FIG. 4 (a)) over the whole area of BP waveform (Aₛ+A_{d} in FIG. 4) within a cardiac cycle.

### Run1: Scenario Adaptive SVA-AS Model

In some embodiments, to simplify the calculation of SVA, the ratio of the blood pressure waveform area of Equation (1) may be modeled as the function of the systolic duration (Ts) and cardiac duration (Tc) with pulse wave information as input parameters, i.e.,

$$SVA {=}^{△} ( δ + Δ {δ}_{pulse} ) \cdot ( 1 - \frac{Ts}{Tc} ) \qquad ( Equation ( 2 ) )$$

$$wherein$$

$$δ = \frac{DBP}{MBP} \cdot κ ,$$

κ is a constant form factor which is the ratio of the area of diastolic BP curve and rectangular area of DBP in the diastolic duration and can be computed by a one-shot BP measurement. In some embodiments, δ can be expressed as

$$δ = \frac{DBP + MBP}{2 \cdot DBP} ,$$

and thus can be related to the ratio of DBP to MBP.

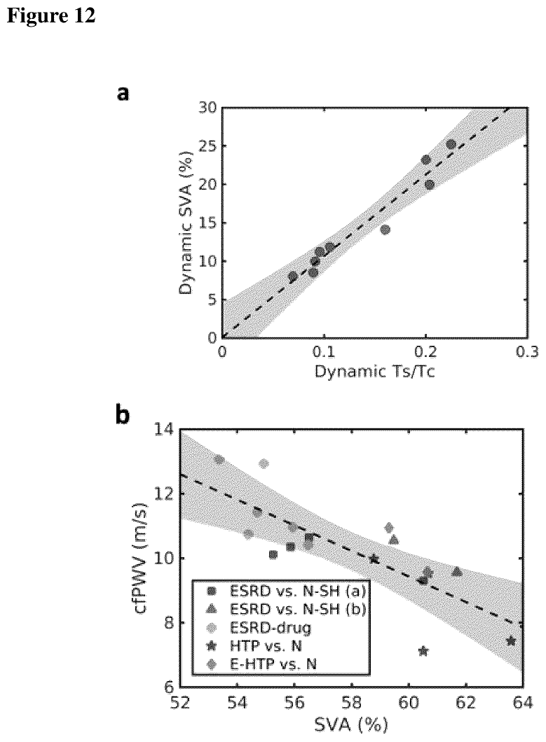

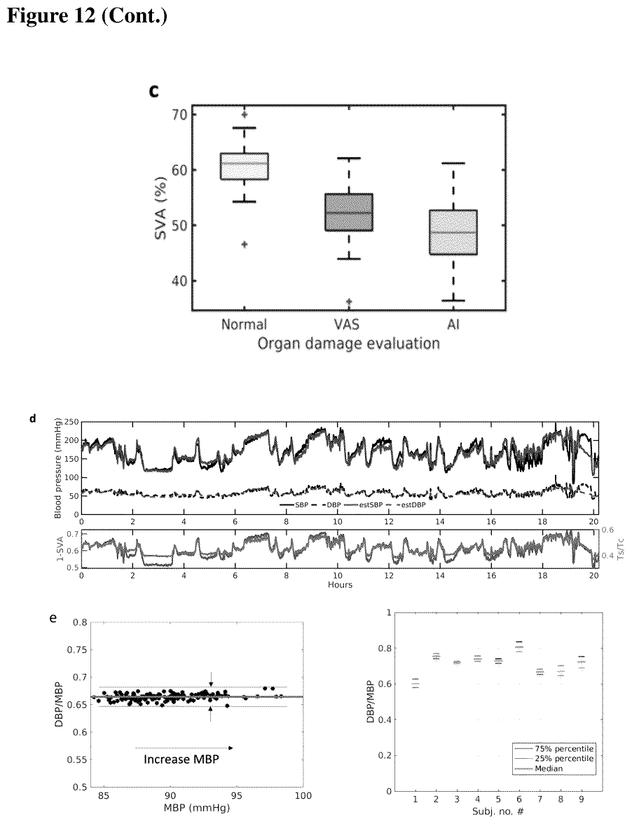

FIG. 12 (e) shows that the ratio of DBP to MBP is stable for each subject (left panel, based on data collected from a patient with myocardial infarction at Prince of Wales Hospital (PWH), and the ratio of DBP to MBP varies among different subjects (right panel, based on data from the MIMIC-I dataset in Example 1).

Δδₚᵤₗₛₑ is the compensation term of the pulse wave-based SVA. This compensation term describes various arterial statuses that associate with different SVA values (e.g., the statuses in FIG. 6 (a-b)). In some embodiments, Δδₚᵤₗₛₑ is a value that represents the relative change in the pulse waveform measured over one cardiac cycle while the subject is at the test state to the pulse waveform measured when the subject is at the resting state. In some embodiments,

$$Δ {δ}_{pulse} = \frac{{Ad}_{testing} - {Ad}_{resting}}{{Ad}_{resting}} .$$

The Ad_{testing} is the area under a pulse waveform during the diastolic phase of the cardiac cycle when the subject is at a test status, and the Ad_{resting} is the area under a pulse waveform during the diastolic phase of a cardiac cycle when the subject is at a resting status. The pulse waveforms relating to Ad_{testing} and/or Ad_{resting} can be determined by a pulse sensor. In some particular embodiments, Δδₚᵤₗₛₑ is associated with the arterial status of the subject and can be assigned with a predetermined value after the arterial status of the subject is determined. For example, a wearable device can record various predetermined values of Δδₚᵤₗₛₑ, and can determine the current arterial status of the subject and assign a corresponding value of Δδₚᵤₗₛₑ. For example, the wearable device can detect that the subject is exercising (e.g., through the subject's heart rate) and determine that the subject is at a status of vasodilation.

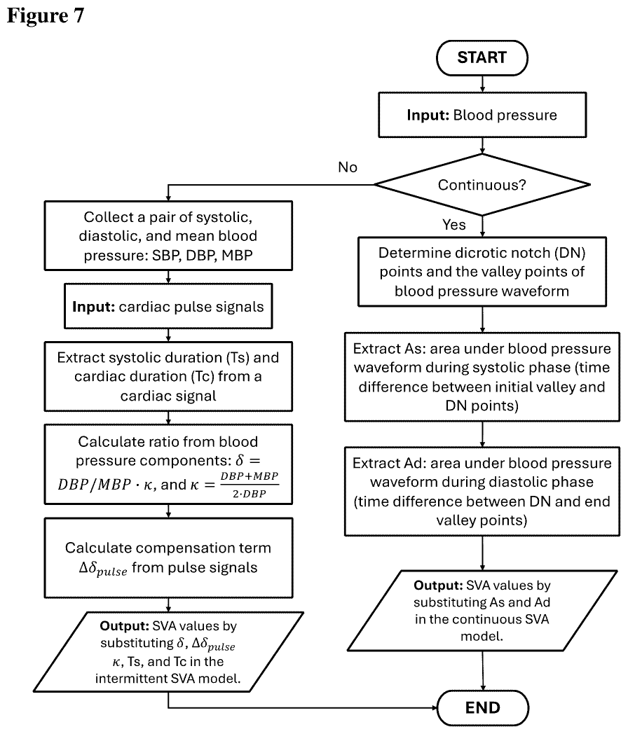

An exemplary workflow of SVA calculation is shown in FIG. 7.

In addition, in some embodiments, the SVA stiffness gradient (shown in FIG. 6 (c-d)) between the central (SVAc) and peripheral (SVAp) arteries can be expressed as follows.

Ratio_{SVA}=SVAₚ/SVA_{c} (Equation (3)).

It is well known in the art that blood pressure waveforms acquired at different vascular segments with different degrees of stiffness are different (see, e.g., Michel E. Safar and Harry Struijker Boudier, Vascular Development, Pulse Pressure, and the Mechanisms of Hypertension. Hypertension. Volume 46, Issue 1, 1 Jul. 2005, pages 205-209). The differences in blood pressure waveforms result in different SVA values. Therefore, RatiosVA can be used to indicate the degree of arterial stiffness in the peripheral artery of the subject. An increase in the RatioSVA value in the subject as compared with a reference RatioSVA value derived from a healthy individual indicates a higher degree of arterial stiffness in the peripheral artery of the subject than the healthy individual. In some embodiments, the reference RatioSVA value can be derived from a population of healthy individuals, and an increase in the RatioSVA value in the subject as compared with the reference RatioSVA value indicates a higher degree of arterial stiffness in the peripheral artery of the subject than at least one individual from the population of healthy individuals (e.g., at least ¼, ⅓, ½, ⅔, or ¾ of the population).

### Run2: SVA-BP Model

In some embodiments, by introducing the pulse stiffening relationships among systolic, dicrotic notch, and diastolic blood pressure in the scenario adaptive SVA (SVAs) model, SVAs can be linked with BP, i.e.,

$$BP = \frac{{b}_{s}^{*} - SVA \cdot b}{SVA \cdot ( PSR - 1 ) + 1 - {k}_{s}^{*}} \qquad ( Equation ( 4 ) )$$

$$wherein$$

$${b}_{s}^{*} and {k}_{s}^{*} ,$$

are the linear coefficients between DBP and dicrotic notch pressure Pₛ*; PSR (i.e., pulse stiffening ratio) and b are the linear coefficients between DBP and SBP. Note that all these coefficients are observable and stable (>24 hours) as those pressure measures are influenced by similar factors, such as physical activity, respiratory rhythm, and stress. Therefore, in some embodiments, these coefficients can be determined by obtaining a plurality of blood pressure values (e.g., obtainable by a blood pressure measuring device) and SVA values (e.g., calculated according to Equation (1) or Equation (2) descried in above Run1) from a subject (e.g., obtaining at least 4 pairs of values of blood pressure and SVA), and calculating these coefficients with the values, i.e., by substituting the pairs of SVA and BP values with the least square method.

In some embodiments, bₛ*, kₛ*, PSR and b are determined by the following steps:
(1) determining a plurality of pairs of BP and SVA values from blood pressure waveforms and pulse waveforms, wherein a single blood pressure waveform and a single pulse waveform determined at the same timepoint are used to determine each pair of BP and SVA values, and wherein the SVA values can be determined by e.g., Equation (1) or Equation (2);
(2) determining bₛ*, kₛ*, PSR and b by feeding the plurality of pairs of BP and SVA values into a formula for least square method to minimize an error in BP as follows:

$${arg min}_{{b}_{s}^{*} , b , PSR , {k}_{s}^{*}} ‖ BP - \frac{{b}_{s}^{*} - SVA \cdot b}{SVA \cdot ( PSR - 1 ) + 1 - {k}_{s}^{*}} ‖ .$$

As shown in FIG. 12 (e), the ratio of DBP to MBP is almost a constant for a specific individual, and thus

$$δ ( δ = \frac{DBP + MBP}{2 \cdot DBP} )$$

in the equation

$$SVA {=}^{△} ( δ + Δ {δ}_{pulse} ) \cdot ( 1 - \frac{Ts}{Tc} )$$

is almost a constant. As described above, Δδₚᵤₗₛₑ is associated with the arterial status of the subject and can be assigned with a predetermined value after the arterial status of the subject is determined. Therefore, in some embodiments, the SVA calculation is primarily based on the data from a cardiac rhythm measurement (including Ts and Tc). BP values (including DBP and MBP) are usually taken as a baseline for calibrating the SVA. The measurement of the BP values is typically done using a home or hospital cuff device (e.g., the Omron blood pressure monitor from Omron, Inc.). Once this initial calibration is complete, the data from a cardiac rhythm measurement is used to calculate SVA in real time. This allows the measurement of dynamic BP continuously based on the real-time SVA. In other words, BP changes in real time based on SVA variation. In some embodiments, BP is related to

$$SVA {=}^{△} ( δ + Δ {δ}_{pulse} ) \cdot ( 1 - \frac{Ts}{Tc} ) ,$$

e.g., as shown in FIG. 14(d).

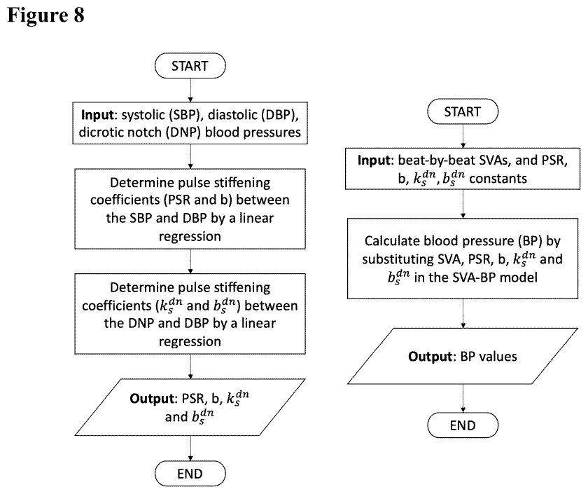

An exemplary workflow of scenario adaptive SVA-BP model is shown in FIG. 8.

### Run3: SVA-CVD Model

In some embodiments, the SVA-CVD model is constructed by a logistic regression model where the input vital signs involved SVA, BP, heart rate (HR), and left ventricular ejection time (LVET). The output probability of abnormal CVD is expressed by a logistic regression model:

p(x)=1−1/(1+e^{-(β}^{₀}^{+β}^{₁}^{x}^{₁}^{+β}^{₂}^{x}^{₂}^{+β}^{₃}^{x}^{₃}^{+β}^{₄}^{x}^{_{4+β}}^{₅}^{x}^{₅}⁾) (Equation (5).

X₁ is a heart rate (HR), X₂ is a left ventricular ejection time (LVET), X₃ is a systolic blood pressure (SBP), X₄ is a diastolic blood pressure (DBP), X₅ is a SVA value, and β₀, β₁, β₂, β₃, β₄, and β₅ are constants. Notably, β₀, β₁, β₂, β₃, β₄, and β₅ can be optimised based on data from healthy subjects and patients with a cardiovascular disease. In some embodiments, for one or more subjects, β₀=−92.7477, β₁=0.3109, β₂=41.5207, β₃=0.0627, β₄=−0.1047, β₅=0.9826.

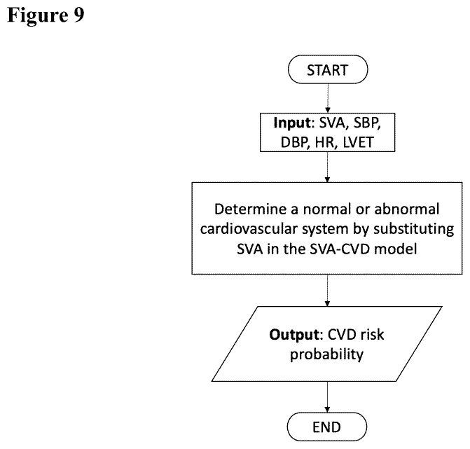

An exemplary workflow of SVA-CVD model is shown in FIG. 9.

### Run4: SRC-OD Model

In some embodiments, the SRC-OD model is constructed by a SRC calculation model and the related cardiovascular system (CVS) indicators where the input vital signs involved SVA, BP, heart rate (HR which is equal to 1/Tc), and left ventricular ejection time (LVET which is equal to Ts). For example, the SRC may be calculated by Equation (6):

$$SRC = 2 \frac{SVA}{\frac{( {e}^{\frac{{T}_{c} - {T}_{s}}{τ}} - 1 ) \cdot DBP}{PP}} - 1. \qquad ( Equation ( 6 ) )$$

τ is the time constant that can be extracted from the pulse waveform and the ratio of DBP to PP. In some embodiments, τ can be calculated by the equation:

$$\frac{DBP}{PP} = \frac{{e}^{\frac{- {T}_{s}}{τ}} \cdot \frac{1}{{( \frac{1}{τ} )}^{2} + {( \frac{π}{{T}_{s}} )}^{2}} [ ( {e}^{\frac{{T}_{s}}{τ} + 1} ) {( \frac{π}{{T}_{s}} )}^{2} ]}{2 \cdot ( {e}^{\frac{- {T}_{c} - {T}_{s}}{τ}} - {e}^{\frac{- {T}_{s}}{τ}} )} .$$

Then SRC can be calculated by Equation (6) where the SVA can be calculated by Equations (1) and (2).

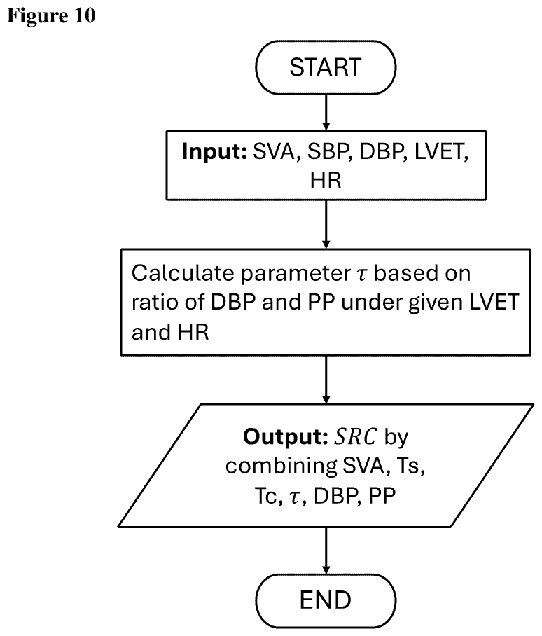

An exemplary workflow of SRC calculation is shown in FIG. 10.

In some embodiments, CVS indicators may be further constructed by Equations (7-11) as follows, where each parameter \tilde{#}represents a value measured for the subject relative to a reference value. The reference value can be derived from a population of healthy individuals, as described above.

NormI= [symbol] · [symbol] / [symbol] (Equation (7));

PASI= [symbol] / [symbol] (Equation (8));

PODI= [symbol] / [symbol] · [symbol] (Equation (9));

HRMDI= [symbol] · [symbol] · [symbol] (Equation (10));

HZDI=1/( [symbol] · [symbol] ) (Equation (11)).

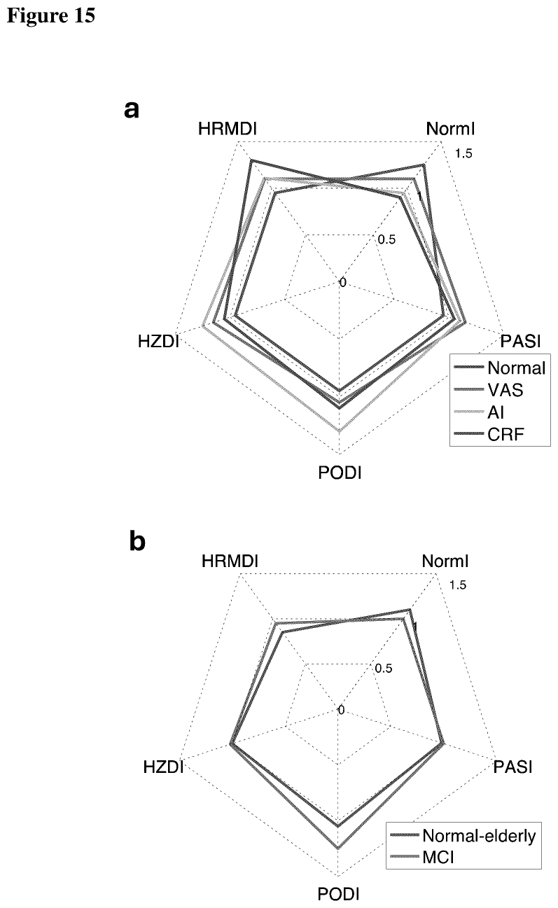

In some embodiments, as shown in FIG. 15 (a), healthy subjects have larger NormI values and smaller PASI, PODI, HRMDI and HZDI values than patients with aortic incompetence (AI), patients with valvular aortic stenosis (VAS), or patients with chronic renal failure (CRF). In some embodiments, as shown in FIG. 15 (b), healthy subjects have larger NormI values and smaller PASI, PODI, HRMDI and HZDI values than patients with mild cognitive impairment (MCI).

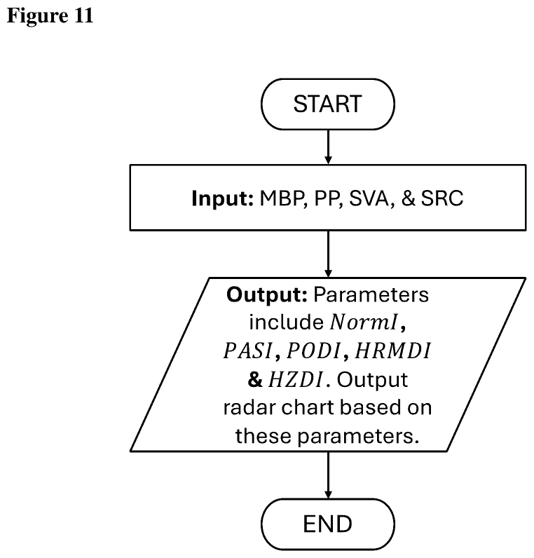

An exemplary workflow of the CVS indicators is shown in FIG. 11.

### EXAMPLES

The present disclosure will be further illustrated below in connection with specific Examples. It is to be understood that these Examples are merely illustrative of the present disclosure and are not intended to limit the scope of the present disclosure.

### Example 1: SVA Validation in Open Clinical Datasets

This example presents SVA validations among open clinical datasets. The datasets included:
(a) 9 ICU patients in the MIMIC-I dataset for exploring the relationship between dynamic SVA and dynamic Ts/Tc, where Ts is the systolic duration, and Tc is the heartbeat cycle (cardiac duration) conducted on;
(b) cross-sectional studies, which included population groups of E-HPT (essential hypertension), N—SH (normal but with systolic hypertension), HPT (hypertension), ESRD (end-stage renal disease), and N (normal subject without diseases), for exploring the relationship between SVA and cfPWV implied SVA;
(c) three datasets for evaluation of SVA in CHD assessment: 1) the normal, 2) valvular aortic stenosis (VAS), and 3) aortic incompetence (AI);
(d) an ICU patient in the MIMIC-I dataset for the application of SVA for BP tracking. The results of SVA validations among open clinical datasets are shown in FIG. 12. SVA values in FIG. 12(a, d) were determined with

$$SVA = \frac{{SV}_{c}}{SV} = \frac{Ad}{As + Ad} . \qquad ( Equation ( 1 ) )$$

SVA values in FIG. 12(b, c) were determined with

$${SVA}_{s} {=}^{Δ} ( δ + Δ {δ}_{pulse} ) \cdot ( 1 - \frac{Ts}{Tc} ) ,$$

as described in Run1.

FIG. 12(a, d) demonstrate that dynamic SVA values can be determined with Equation (1). FIG. 12(c) shows significant SVA differences among the normal individuals, individuals with valvular aortic stenosis (VAS), and individuals with aortic incompetence (AI). The results in FIG. 12(c) demonstrate that SVA values can be used to evaluate organ damages in the subjects. This indicates the potential clinical value of the SVA measurement in arterial system risk assessment especially for subjects with poor arterial elastic system caused by cardiovascular diseases.

### Example 2: Application of Integrated Wearable Cardiac Sensors in SVA Measurement & Validation of SVA-BP Tracking, SRC Measurement, and Indicators Derived From SVA

### 2.1 Application of Integrated Wearable Cardiac Sensors in SVA Measurement

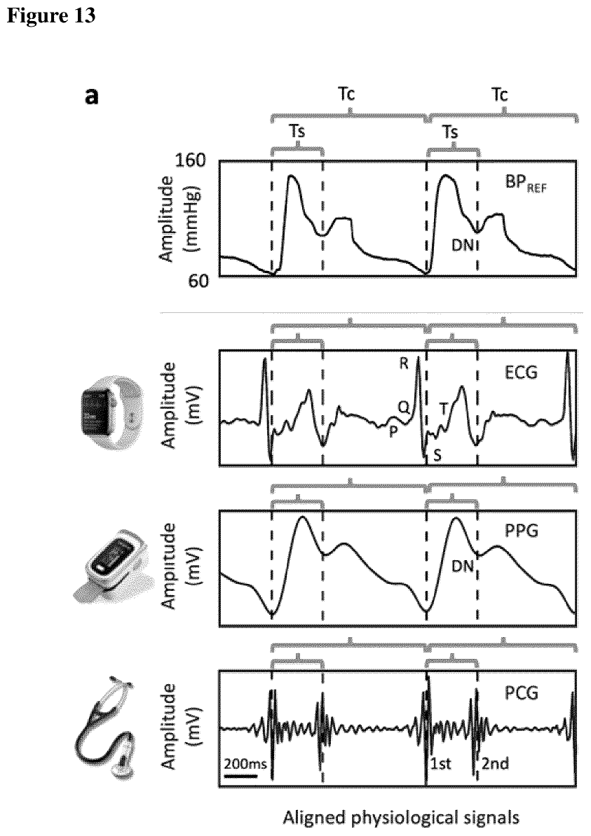

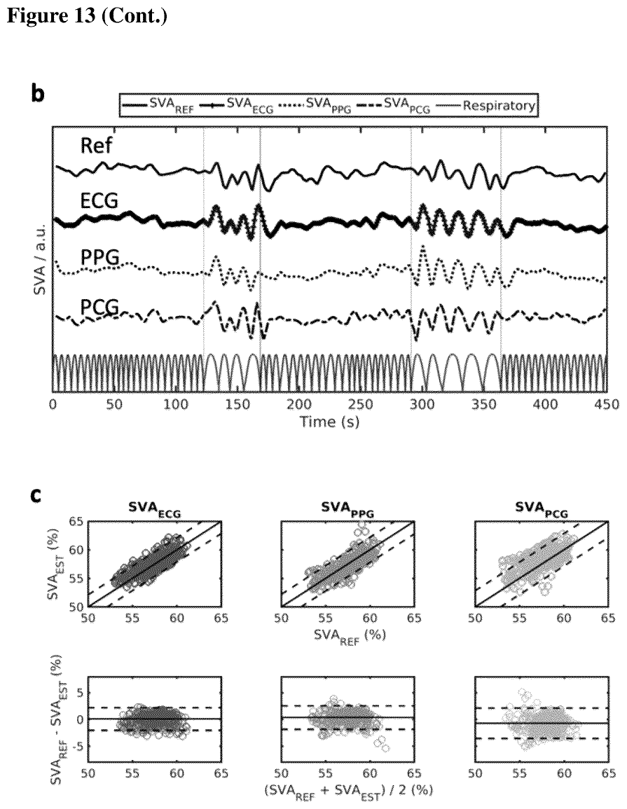

In this example, the application of integrated wearable cardiac sensors in SVA measurement is presented. Results are shown in FIG. 13. The cardiac information which included the systolic duration and heartbeat cycle were measurable by various wearable cardiac sensors such as pressure sensors, ECG, PCG and PPG at the same subjects, shown in FIG. 13(a). The dynamic SVA trend was traced by different cardiac sensors as shown in FIG. 13(b). The comparisons of accuracy of the SVA extracted from different cardiac sensors were shown in FIG. 13(c). In FIG. 13, SVA_{REF} was determined with Equation (1)

$$SVA = \frac{{SV}_{c}}{SV} = \frac{Ad}{As + Ad} ,$$

while other SVA values were determined with Equation (2)

$${SVA}_{s} {=}^{Δ} ( δ + Δ {δ}_{pulse} ) \cdot ( 1 - \frac{Ts}{Tc} ) ,$$

as described in Run1, and values of DBP, MBP, and Δδₚᵤₗₛₑ were average values. In FIG. 13(b, c), SVA_{EST} includes SVA_{ECG}, SVA_{PPG}, and SVA_{PCG}, which were calculated with Ts and Tc values extracted from ECG signals, PPG signals, PCG signals, respectively. As shown in FIG. 13(b, c), any one of SVA_{ECG}, SVA_{PPG}, and SVA_{PCG} is consistent with SVA_{REF}. This indicates that Equation (1) and Equation (2) can both be used to calculate SVA values.

The demonstration of off-the-shelf wearable devices in SVA and SRC measurement and the related CVS assessment in daily life was shown in FIGS. 14, 15 and 16. SVA values used to generate the results in FIGS. 14, 15 and 16 were determined

$${SVA}_{s} {=}^{Δ} ( δ + Δ {δ}_{pulse} ) \cdot ( 1 - \frac{Ts}{Tc} ) ,$$

as described in Run1. ECG-based Apple watch and PPG-based INTS finger ring were explored in these tasks (FIG. 14(a)). Short-term ECG measurement (~30 seconds) by Apple watch was conducted on the subjects with and without coronary arterial disease (CAD). Simultaneously, the Vicorder system was performed to measure the gold standard cfPWV in arterial stiffness. The consistency trend of the ECG-based & PPT-based 1-SVA and cfPWV in FIG. 14(b), implied the SVA can be used in wearable device as a vascular age probe. Furthermore, in FIG. 14(c), the significant differences of the SVA between the CAD patients who had undergone percutaneous coronary intervention (PCI) (34 patients) and normal subjects (14 normal subjects) were observed which indicated the off-the-shelf ECG-based Apple watch was acceptable in AS assessment.

### 2.2 Validation of SVA-BP Tracking

Besides, FIG. 14(d) demonstrated the application of INTS finger ring by PPG-based SVA-BP tracking. The SVA-BP model by PPG ring was performed on 13 elderly people whose heart rhythm could be clearly extracted from the PPG signals. The high accuracy of BP estimation by SVA-BP model was shown in the FIG. 14(e) which indicated the off-the-shelf PPG-based finger ring was workable in daily BP tracking. These results indicate that SVA measurements can be used to track blood pressure.

### 2.3 Application of SVA in Determining Probability of Cardiovascular Diseases

FIG. 14 (f) shows the CHD assessment by the statistic distinguishing normal and poor arterial systems (i.e., marked CVDs) between the total datasets with and without using wearable device measurement. Data was acquired from 41 subjects with the CHD and 104 subjects without the CHD. The SVA showed high relevance in the classification of arterial system while BP seemed to be a poor indicator in CHD assessment.

FIG. 14 (g) shows the validation of p(x) scores derived from SVA measurements in determining probability of cardiovascular diseases, in which p(x) scores were determined with Equation (5): p(x)=1−1/(1+e^{-(β}^{₀}^{+β}^{₁}^{x}^{₁}^{+β}^{₂}^{x}^{₂}^{+β}^{₃}^{x}^{₃}^{+β}^{₄}^{x}^{_{4+β}}^{₅}^{x}^{₅}⁾), and SVA values fed into Equation (5) were determined with Equation (2)

$${SVA}_{s} {=}^{Δ} ( δ + Δ {δ}_{pulse} ) \cdot ( 1 - \frac{Ts}{Tc} ) .$$

Data was acquired from 120 subjects, including 36 healthy individuals, 30 patients with aortic incompetence (AI), 40 patients with valvular aortic stenosis (VAS), and 14 patients with chronic renal failure (CRF). In FIG. 14(g), subjects with p(x) scores of above 0.5 (i.e., from 0.5 to 1) were classified as healthy individuals, while subjects with p(x) scores of less than 0.5 were classified as diseased individuals. The “Ground truth” line represents actual condition of the 120 subjects (healthy or diseased). As shown in FIG. 14(g), p(x) scores derived from SVA measurements can accurately determine the probability of cardiovascular diseases of individuals.

### 2.4 Validation of SRC Measurements

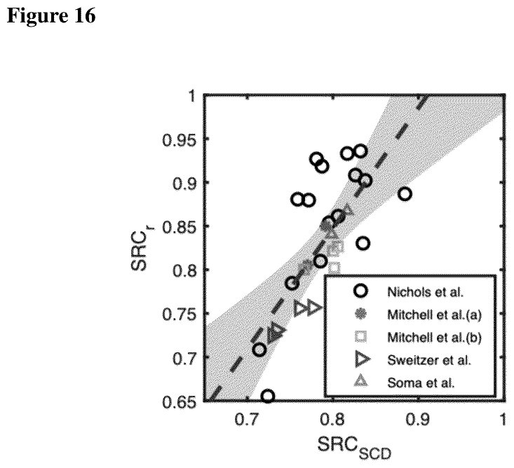

FIG. 16 shows results of the validation of SRC measurements. In FIG. 16, SRC_{SCD} values of the present application were calculated by Equation (6) with SVA values, where the SVA values were calculated by Equation (2) with Ts/Tc in subjects with organ damages. Reference SRC (SRCr) values can be determined by performing a Fourier analysis of data relating to arterial blood pressure and arterial blood flow acquired at the same arterial segment. In this example, SRCr values were extracted from results from five research groups as follows.

Nichols et al. (Nichols et al. Input impedance of the systemic circulation in man. Circ. Res. 40, (1977)) represents data from 16 individuals, including 5 normal subjects and 11 patients with coronary heart disease.

Mitchell et al. (a) (Mitchell, G. F. et al. Pulsatile hemodynamics in congestive heart failure. Hypertens. Dallas Tex 1979 38, 1433-1439 (2001)) represents data from 68 individuals divided into two groups, including 28 normal subjects and 40 patients with heart failure.

Mitchell et al. (b) (Mitchell, G. F. et al. Omapatrilat reduces pulse pressure and proximal aortic stiffness in patients with systolic hypertension: results of the conduit hemodynamics of omapatrilat international research study. Circulation 105, 2955-2961 (2002)) represents data from 158 individuals divided into four groups, including normal subjects and patients with hypertension.

Sweitzer et al. (Sweitzer, N. K. et al. Left Ventricular Responses to Acute Changes in Late Systolic Pressure Augmentation in Older Adults. Am. J. Hypertens. 26, 866-871 (2013)) represents data from 16 individuals divided into four groups, including elderly individuals with hypertension.

Soma et al. (Soma, J., Angelsen, B. A. J., Techn, D., Aakhus, S. & Skjærpe, T. Sublingual Nitroglycerin Delays Arterial Wave Reflections Despite Increased Aortic “Stiffness” in Patients with Hypertension: A Doppler Echocardiography Study. J. Am. Soc. Echocardiogr. 13, 1100-1108 (2000)) represents data from 66 individuals divided into two groups of individuals with hypertension.

As shown in FIG. 16, SRC_{SCD} values in the present application are consistent with Reference SRC (SRCr) values, demonstrating that the SRC measurements in the present application can accurately indicate an organ damage in individuals.

2.5 Validation of Indicators Derived from SVA

Further, subjects with diseases including VAS, AI, MCI and CRF, and normal individuals were enrolled. Indicators derived from SVA, including NormI, PASI, PODI, HRMDI and HZDI, were calculated with Equation (7) to Equation (11) in Run4, respectively. Results are shown in FIG. 15. FIG. 15 showed the SRC assessment for different types of diseases that were associated with the heart and aorta (VAS, AI), brain (MCI), and kidney (CRF) etc. As shown in FIG. 15(a), normal subjects have larger NormI values and smaller PASI, PODI, HRMDI and HZDI values than patients with aortic incompetence (AI), patients with valvular aortic stenosis (VAS), or patients with chronic renal failure (CRF). As shown in FIG. 15(b), normal subjects have larger NormI values and smaller PASI, PODI, HRMDI and HZDI values than patients with mild cognitive impairment (MCI). These results indicate the potential value of NormI, PASI, PODI, HRMDI and HZDI in cardiovascular disease assessment.

### Example 3: Large-Scale Clinical Data Testing

Large-scale clinical data testing involving a hybrid dataset taken from 6 hospitals/research institutes (9 open databases and 4 self-built databases from 878 subjects in total) and diverse measurement approaches was carried out to validate the SVA model. The results showed that the SVA-based parameters correlated well with the gold-standard measurements in arterial stiffness and blood pressure and outperformed the commonly used vital sign (e.g., blood pressure) alone in detecting abnormalities in cardiovascular systems.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or”, and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on”.

The claims may be drafted to exclude any element which may be optional. Aₛ such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted to be prior art. Where a conflict exists between the instant application and a reference provided herein, the instant application shall dominate.

What is claimed is:

1. A system for assessing risk and/or severity of a cardiovascular disease in a subject, comprising:
a blood pressure measuring device and/or a cardiac rhythm monitor, wherein the blood pressure measuring device is configured to perform a blood pressure measurement on the subject, and the cardiac rhythm monitor is configured to perform a cardiac rhythm measurement on the subject;
a processor configured to (i) determine, from data from the blood pressure measurement and/or the cardiac rhythm measurement in the subject, a plurality of stroke volume allocation (SVA) values, (ii) calculate an average SVA value from the plurality of SVA values and compare the average SVA value with a reference SVA value derived from a population of healthy individuals, and (iii) for an average SVA value which is below the reference SVA value, generate an assessment result indicating that the subject has an increased risk and/or severity of the cardiovascular disease as compared with the healthy individuals; and
an output device (e.g. a display) for outputting the assessment result;
wherein an SVA value represents a ratio of a stroke volume stored in the aorta of the subject during the systole phase in a cardiac cycle to a total stroke volume pumped from the heart of the subject during the cardiac cycle.

2. The system of claim 1, wherein the data is from a blood pressure measurement and comprises a blood pressure waveform in the cardiac cycle, and an SVA value is generated by:
i) calculating the area under the blood pressure waveform during the diastolic phase of the cardiac cycle, defined as A_{d};
ii) calculating the area under the blood pressure waveform during the systolic phase of the cardiac cycle, defined as Aₛ; and
iii) calculating the SVA value by the following equation:
$$SVA = \frac{Ad}{As + Ad} .$$

3. The system of claim 2, wherein the blood pressure waveform comprises a first valley point, a dicrotic notch point and a second valley point, the systolic phase is a phase substantially between the first valley point and the dicrotic notch point, and the diastolic phase is a phase substantially between the dicrotic notch point and the second valley point; and
wherein the Aₛ is obtained by calculating the area under the blood pressure waveform from the first valley point to the dicrotic notch point, and the A_{d} is obtained by calculating the area under the blood pressure waveform from the dicrotic notch point to the second valley point.

4. The system of claim 1, wherein the data is from a blood pressure measurement and a cardiac rhythm measurement, and comprises a blood pressure waveform and a pulse waveform in the cardiac cycle, and an SVA value is generated by:
a) extracting a diastolic blood pressure (DBP) and a mean blood pressure (MBP) from the data, and extracting a cardiac duration and a systolic duration from the pulse waveform; and
b) calculating the SVA value by the following equation:
$${SVA}_{s} {=}^{Δ} ( δ + Δ {δ}_{pulse} ) \cdot ( 1 - \frac{Ts}{Tc} ) ,$$
wherein Tₛ is the systolic duration,
T_{c} is the cardiac duration,
$$δ = \frac{DBP + MBP}{2 \cdot DBP} ,$$
$$Δ {δ}_{pulse} = \frac{{Ad}_{testing} - {Ad}_{resting}}{{Ad}_{resting}} .$$
wherein the Ad_{testing} is the area under a pulse waveform during the diastolic phase of the cardiac cycle when the subject is at a test status, and the Ad_{resting} is the area under a pulse waveform during the diastolic phase of a cardiac cycle when the subject is at a resting status.

5. The system of claim 4, wherein the test status is selected from the group consisting of a resting status, a status of vasodilation, a status of vasoconstriction and any combination thereof.

6. The system of claim 1, wherein the blood pressure measurement and/or the cardiac rhythm measurement are performed on a peripheral artery and a central artery, respectively; and
wherein the processor is further configured to:
A) determine, from data from the blood pressure measurement and/or the cardiac rhythm measurement in the subject, a first SVA value for the peripheral artery and a second SVA value for the central artery;
B) determine RatioSVA as a ratio of the first SVA value to the second SVA value, and
C) assess arterial stiffness with the RatioSVA; and
wherein an increase in the RatioSVA value in the subject as compared with a reference Ratio_{SVA} value derived from a healthy individual indicates a higher degree of arterial stiffness in the peripheral artery of the subject than the healthy individual.

7. The system of claim 4, wherein the processor is further configured to:
(1) by using the steps a) and b) in claim 4, generate a plurality of pairs of blood pressure (BP) and SVA values and calculate an average δ at a plurality of first timepoints, wherein a single blood pressure waveform and a single pulse waveform obtained at the same timepoint are used to determine each pair of BP and SVA values,
(2) determine bₛ*, kₛ*, PSR and b by feeding the plurality of pairs of BP and SVA values into a formula for least square method to minimize an error in BP as follows:
$${argmin}_{{b}_{s}^{*} , b , PSR , {k}_{s}^{*}} ‖ BP - \frac{{b}_{s}^{*} - SVA \cdot b}{SVA \cdot ( PSR - 1 ) + 1 - {k}_{s}^{*}} ‖ ,$$
(3) generate SVA values at a plurality of second timepoints from a pulse waveform in the cardiac cycle by the following equation:
$${SVA}_{s} {=}^{Δ} ( δ + Δ {δ}_{pulse} ) \cdot ( 1 - \frac{Ts}{Tc} ) ,$$
wherein Tₛ, T_{c}, and Δδₚᵤₗₛₑ are defined as in claims 4 and 8 is the average δ calculated in step (1); and
(4) calculate BP values through the SVA values at the plurality of second timepoints according to the following equation:
$$BP = \frac{{b}_{s}^{*} - SVA \cdot b}{SVA \cdot ( PSR - 1 ) + 1 - {k}_{s}^{*}} .$$

8. The system of claim 1, wherein the processor is further configured to determine a probability of the cardiovascular disease by the following equation:
p(x)=1−1/(1+e^{-(β}^{₀}^{+β}^{₁}^{x}^{₁}^{+β}^{₂}^{x}^{₂}^{+β}^{₃}^{x}^{₃}^{+β}^{₄}^{x}^{_{4+β}}^{₅}^{x}^{₅}⁾),
wherein X₁ is a heart rate (HR), X₂ is a left ventricular ejection time (LVET), X₃ is a systolic blood pressure (SBP), X₄ is a diastolic blood pressure (DBP), X₅ is a SVA value, β₀=−92.7477, β₁=0.3109, β₂=41.5207, β₃=0.0627, β₄=−0.1047, β₅=0.9826, and
wherein an increase in the p(x) value in the subject as compared with a reference p(x) value derived from a population of healthy individuals indicates that the subject has a risk of developing the cardiovascular disease.

9. The system of claim 1, wherein the data is from a blood pressure measurement and a cardiac rhythm measurement, and the processor is further configured to assess an organ damage induced by a pulsatile energy in the cardiovascular system of the subject with a systemic reflection coefficient (SRC) value calculated by the following equation:
$$SRC = 2 \frac{SVA}{\frac{( {e}^{\frac{{T}_{c} - {T}_{s}}{τ}} - 1 ) \cdot DBP}{PP}} - 1 ,$$
wherein DBP is a diastolic blood pressure, PP is a pulse pressure, Tₛ is a systolic duration from a pulse waveform, T_{c} is a cardiac duration from the pulse waveform, and wherein DBP and PP are from the blood pressure measurement, Tₛ and T_{c} are from the cardiac rhythm measurement, and τ is calculated with DBP, PP, Ts and Tc by the following equation:
$$\frac{DBP}{PP} = \frac{{e}^{\frac{- {T}_{s}}{τ}} \cdot \frac{1}{{( \frac{1}{τ} )}^{2} + {( \frac{π}{{T}_{s}} )}^{2}} [ ( {e}^{\frac{{T}_{s}}{τ} + 1} ) {( \frac{π}{{T}_{s}} )}^{2} ]}{2 \cdot ( {e}^{\frac{- {T}_{c} - {T}_{s}}{τ}} - {e}^{\frac{- {T}_{S}}{τ}} )} ;$$
and wherein an increase in the SRC value in the subject as compared with a reference SRC value derived from a population of healthy individuals indicates an organ damage in the subject.

10. The system of claim 9, wherein the processor is further configured to assess the cardiovascular disease with indicators derived from the SRC, the SVA, a mean blood pressure (MBP) and the PP as follows:
NormI= [symbol] · [symbol] / [symbol] ;
PASI= [symbol] / [symbol] ;
PODI= [symbol] / [symbol] · [symbol] ;
HRMDI= [symbol] · [symbol] · [symbol] ;
HZDI=1/( [symbol] · [symbol] );
wherein the symbol ~ for each parameter represents a ratio of a value measured for the subject to a reference value derived from a population of healthy individuals; and
wherein an increase in NormI in the subject from a first timepoint to a second timepoint indicates an improvement in the cardiovascular system of the subject from the first timepoint to the second timepoint,
an increase in PASI in the subject from a first timepoint to a second timepoint indicates a higher risk for arterial stiffness due to poor cushioning function and a higher SRC in the subject at the first timepoint than that at the second timepoint,
an increase in PODI in the subject from a first timepoint to a second timepoint indicates a higher risk for pulsatile-induced organ damage due to poor pulsation reflexes, poor cushioning function, and a large pulsation component of BP in the subject at the first timepoint than that at the second timepoint,
an increase in HRMDI in the subject from a first timepoint to a second timepoint indicates a higher risk for systemic-vascular-resistance-induced microcirculation damage in the subject at the first timepoint than that at the second timepoint,
an increase in HZDI in the subject from a first timepoint to a second timepoint indicates a higher risk for hazardous cardiovascular system with arterial stiffness and pulsatile-induced organ damage in the subject at the first timepoint than that at the second timepoint.

11. The system of claim 1, wherein the cardiovascular disease is selected from the group consisting of mild cognitive impairment, stroke, dementia, valvular aortic stenosis, aortic incompetence and any combination thereof.

12. The system of claim 1, wherein the assessment result comprises an alert about the risk and/or severity of the cardiovascular disease, and/or a treatment recommendation.

13. The system of claim 1, further comprising:
a memory configured to store data from the blood pressure measurement and/or the cardiac rhythm measurement, the reference SVA value and/or the assessment result.

14. The system of claim 1, wherein the blood pressure measuring device is selected from the group consisting of a pressure sensor, a digital sphygmomanometer, stethoscope sphygmomanometer and any combination thereof; and/or
the cardiac rhythm monitor is selected from the group consisting of a cardiac rhythm monitor based on electrocardiogram (ECG), a cardiac rhythm monitor based on photoplethysmography (PPG), a cardiac rhythm monitor based on phonocardiography (PCG), a cardiac rhythm monitor based on impedance cardiography (ICG), a cardiac rhythm monitor based on ultrasound, a cardiac rhythm monitor based on ballistocardiography (BCG), a cardiac rhythm monitor based on seismocardiographic (SCG) and any combination thereof.

15. The system of claim 1, wherein the blood pressure measuring device and the cardiac rhythm monitor are configured to make simultaneous measurements.

16. A method for assessing risk and/or severity of a cardiovascular disease in a subject, comprising:
generating data from a blood pressure measurement and/or a cardiac rhythm measurement in the subject;
determining from the data a plurality of stroke volume allocation (SVA) values;
calculating an average SVA value from the plurality of SVA values and comparing the average SVA value with a reference SVA value derived from a population of healthy individuals;
for an average SVA value which is below the reference SVA value, generating an assessment result indicating that the subject has an increased risk and/or severity of the cardiovascular disease as compared with the healthy individuals; and
outputting the assessment result;
wherein an SVA value represents a ratio of a stroke volume stored in the aorta of the subject during the systole phase in a cardiac cycle to a total stroke volume pumped from the heart of the subject during the cardiac cycle.

17. A computer-readable storage medium having stored therein program code instructions that, when executed by a processor in a computer system, cause the processor to perform a method according to claim 16.

18. A computer product comprising a non-transitory computer readable medium storing a plurality of instructions that when executed cause a computer system to perform a method according to claim 16.

19. A system comprising:
the computer product of claim 18; and
one or more processors for executing instructions stored on the computer readable medium.

20. A system comprising one or more processors configured to perform a method according to claim 16.
